(19) United States
(12) Patent Application Publication
(10) Pub. No.: US 2008/0313769 A9
(43) Pub. Date: Dec. 18, 2008

(54) EPSP synthase domains conferring glyphosate resistance

(75) Inventors: Brian Carr, Raleigh, NC (US); Philip E. Hammer, Cary, NC (US); Todd K. Hinson, Rougemont, NC (US); Brian Vande Berg, Durham, NC (US)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: Athenix Corporation, Durham, NC (US)

(21) Appl. No.: 11/651,752

(22) Filed: Jan. 10, 2007

Related U.S. Application Data
(60) Provisional application No. 60/758,320, filed on Jan. 12, 2006.

(51) Int. Cl. A01H 1/00; C07H 21/04; C12N 9/10; C12N 15/82; C12N 5/04

(52) U.S. Cl. 800/278; 435/468; 435/419; 435/193; 536/23.2

(57) ABSTRACT

Compositions and methods for conferring tolerance to glyphosate in bacteria, plants, plant cells, tissues and seeds are provided. Compositions include novel EPSP synthase enzymes and nucleic acid molecules encoding such enzymes, vectors comprising those nucleic acid molecules, and host cells comprising the vectors. The novel proteins comprise at least one sequence domain selected from the domains provided herein. These sequence domains can be used to identify EPSP synthases with glyphosate resistance activity.

### CROSS REFERENCE TO RELATED APPLICATION

This application claims the benefit of U.S. Provisional Application Ser. No. 60/758,320, filed Jan. 12, 2006, the contents of which are herein incorporated by reference in its entirety.

### FIELD OF THE INVENTION

This invention relates to plant molecular biology, particularly to a novel class of EPSP synthases that confer resistance to the herbicide glyphosate.

### BACKGROUND OF THE INVENTION

N-phosphonomethylglycine, commonly referred to as glyphosate, is an important agronomic chemical. Glyphosate inhibits the enzyme that converts phosphoenolpyruvic acid (PEP) and 3-phosphoshikimic acid (S3P) to 5-enolpyruvyl-3-phosphoshikimic acid. Inhibition of this enzyme (5-enolpyruvylshikimate-3-phosphate synthase; referred to herein as “EPSP synthase”, or “EPSPS”) kills plant cells by shutting down the shikimate pathway, thereby inhibiting aromatic amino acid biosynthesis.

Since glyphosate-class herbicides inhibit aromatic amino acid biosynthesis, they not only kill plant cells, but are also toxic to bacterial cells. Glyphosate inhibits many bacterial EPSP synthases, and thus is toxic to these bacteria. However, certain bacterial EPSP synthases have a high tolerance to glyphosate.

Plant cells resistant to glyphosate toxicity can be produced by transforming plant cells to express glyphosate-resistant bacterial EPSP synthases. Notably, the bacterial gene from Agrobacterium tumefaciens strain CP4 has been used to confer herbicide resistance on plant cells following expression in plants. A mutated EPSP synthase from Salmonella typhimurium strain CT7 confers glyphosate resistance in bacterial cells, and confers glyphosate resistance on plant cells (U.S. Pat. Nos. 4,535,060; 4,769,061; and 5,094,945).

U.S. Pat. No. 6,040,497 reports mutant maize EPSP synthase enzymes having substitutions of threonine to isoleucine at position 102 and proline to serine at position 106 (the “TIPS” mutation). Such alterations confer glyphosate resistance upon the maize enzyme. A mutated EPSP synthase from Salmonella typhimurium strain CT7 confers glyphosate resistance in bacterial cells, and is reported to confer glyphosate resistance upon plant cells (U.S. Pat. Nos. 4,535,060; 4,769,061; and 5,094,945). He et al. ((2001) Biochim et Biophysica Acta 1568:1-6) have developed EPSP synthases with increased glyphosate tolerance by mutagenesis and recombination between the E. coli and Salmonella typhimurium EPSP synthase genes, and suggest that mutations at position 42 (T42M) and position 230 (Q230K) are likely responsible for the observed resistance. Subsequent work (He et al. (2003) Biosci. Biotech. Biochem. 67:1405-1409) shows that the T42M mutation (threonine to methionine) is sufficient to improve tolerance of both the E. coli and Salmonella typhimurium enzymes.

Due to the many advantages herbicide resistance plants provide, methods for identifying herbicide resistance genes with glyphosate resistance activity are desirable.

### SUMMARY OF INVENTION

Compositions and methods for conferring resistance or tolerance to glyphosate in bacteria, plants, plant cells, tissues and seeds are provided. Compositions include EPSP synthase enzymes having a Q-loop region with an increased polarity, and nucleic acid molecules encoding such enzymes, vectors comprising those nucleic acid molecules, and host cells comprising the vectors. The EPSP synthase enzymes of the invention comprise at least one sequence domain selected from the following domains: D-C-X₁-X₂-S-G (SEQ ID NO:29), where X₁ denotes glycine, serine, alanine or asparagine, and X₂ denotes asparagine or glutamic acid; or, D-A-X₁-X₂-S-G (SEQ ID NO:30), where X₁ denotes alanine or arginine, and X₂ denotes asparagine or glutamic acid; or, K-L-K-X₁-S-A (SEQ ID NO:31), where X₁ denotes glycine, asparagine or glutamic acid; or, W-C-E-D-A-G (SEQ ID NO:32).

The nucleotide sequences of the invention can be used in DNA constructs or expression cassettes for transformation and expression in organisms, including microorganisms and plants. Compositions also comprise transformed bacteria, plants, plant cells, tissues, and seeds that are glyphosate resistant by the introduction of the compositions of the invention into the genome of the organism. Where the organism is a plant, the introduction of the sequence allows for glyphosate containing herbicides to be applied to plants to selectively kill glyphosate sensitive weeds or other untransformed plants, but not the transformed organism.

Methods for identifying an EPSP synthase with glyphosate resistance activity are additionally provided. The methods comprise obtaining an amino acid sequence for an EPSP synthase and analyzing the Q-loop region increased polarity. Additionally, the amino acid sequence can be analyzed to determine whether the amino acid sequence comprises at least one sequence domain of the invention.

### DESCRIPTION OF FIGURES

FIG. 1 shows an alignment of the amino acid region corresponding to the Q-loop region described herein. The alignment shows GRG1 (amino acid residues 80-100 of SEQ ID NO:2); Clostridium perfringens EPSPS (amino acid residues 80-100 of SEQ ID NO:3); GRG10 (amino acid residues 80-100 of SEQ ID NO:6); GRG21 (amino acid residues 80-100 of SEQ ID NO:8); GRG22 (amino acid residues 80-100 of SEQ ID NO:10); GRG20 (amino acid residues 80-100 of SEQ ID NO:12); GRG23 (amino acid residues 80-100 of SEQ ID NO:14); GRG15 (amino acid residues 80-100 of SEQ ID NO:15); GRG5 (amino acid residues 80-100 of SEQ ID NO:16); GRG12 (amino acid residues 80-100 of SEQ ID NO:17); GRG6 (amino acid residues 80-100 of SEQ ID NO:18); GRG7 (amino acid residues 80-100 of SEQ ID NO:19); GRG8 (amino acid residues 80-100 of SEQ ID NO:20); GRG9 (amino acid residues 80-100 of SEQ ID NO:21); E. coli AroA (amino acid residues 85-106 of SEQ ID NO:22); Salmonella typhimurium EPSPS (amino acid residues 85-106 of SEQ ID NO:23); Zea mays EPSPS (amino acid residues 85-106 of SEQ ID NO:24); Agrobacterium tumefaciens strain CP4 EPSPS (amino acid residues 85-106 of SEQ ID NO:25); Bacillus subtilis AroA (amino acid residues 85-106 of SEQ ID NO:26); and Kleibsella pneumoniae EPSPS (amino acid residues 85-106 of SEQ ID NO:27).

### DETAILED DESCRIPTION OF THE INVENTION

I. Compositions

Compositions and methods for conferring herbicide resistance or tolerance, particularly glyphosate resistance or tolerance, in organisms are provided. The methods involve transforming organisms with nucleotide sequences encoding a glyphosate tolerance gene wherein said gene encodes a polypeptide having a Q-loop comprising an amino acid sequence with increased polarity. The region of the Q-loop can be identified by aligning amino acid sequences with the conserved arginine in the amino acid region corresponding to positions 90-105 of SEQ ID NO:22. As used herein, the phrase “corresponding to” or “corresponds to” when referring to amino acid (or nucleotide) position numbers means that one or more amino acid (or nucleotide) sequences aligns with the reference sequence at the position numbers specified in the reference sequence. For example, to identify a Q-loop region in an amino acid sequence that corresponds to amino acids 90-105 of SEQ ID NO:22, one could align the amino acid sequence in question with the amino acid sequence of SEQ ID NO:22 using alignment methods discussed elsewhere herein, and identify the region of the amino acid sequence in question that aligns with amino acid residues 90-105 of SEQ ID NO:22. It is recognized that the amino acid number may vary by about plus or minus 20, 15, 10, 9, 8, 7, 6, 5, 4, 3, 2 or 1 amino acid(s) on either side of the Q-loop. The region is believed to be involved in the recognition of the substrate PEP. In particular, the present invention recognizes a class of enzymes that confers glyphosate resistance or tolerance, and nucleotide sequences encoding such enzymes. Such enzymes may also be identified by having at least one sequence domain of the invention. By “sequence domain of the invention” is intended at least one domain selected from the following:
D-C-X₁-X₂-S-G (SEQ ID NO:29), where X₁ denotes glycine, serine, alanine or asparagine, and X₂ denotes asparagine or glutamic acid; or,
D-A-X₁-X₂-S-G (SEQ ID NO:30), where X₁ denotes alanine or arginine, and X₂ denotes asparagine or glutamic acid; or,
K-L-K-X₁-S-A (SEQ ID NO:31), where X₁ denotes glycine, asparagine or glutamic acid; or,
W-C-E-D-A-G (SEQ ID NO:32).
   In another embodiment, the sequence domain of the invention further comprises a serine or threonine at the amino acid position corresponding to residue 98 of SEQ ID NO:22. By “increased polarity of the Q-loop region” is intended that one or more of the amino acids within the Q-loop have an increased polarity when compared to the same region of an EPSP synthase not containing a sequence domain of the invention. The sequences find use in preparing plants that show increased resistance to the herbicide glyphosate. Thus, transformed bacteria, plants, plant cells, plant tissues and seeds are provided.

A. EPSP Synthase

In the present invention, the class of enzymes that confers glyphosate resistance is EPSP synthases. The term “EPSP synthase” as used herein refers to both a native EPSP synthase or a variant or fragment thereof. EPSP synthase is involved in the penultimate step in the shikimic acid pathway for the biosynthesis of aromatic amino acids and many secondary metabolites, including tetrahydrofolate, ubiquinone and vitamin K (Gruys et al. (1999) Inhibitors of Tryptophan, Phenyalanine, and Tyrosine Biosynthesis as Herbicides (Dekker, N.Y.)). EPSP synthase converts phosphoenolpyruvic acid (PEP) and 3-phosphoshikimic acid (S3P) to 5-enolpyruvyl-3-phosphoshikimic acid (Amrhein et al. (1980) Plant Physiol. 66:830-834). The monomeric EPSP synthase is one of two enzymes in the class of enolpyruvyltransferases. This class of polypeptides shares a unique structure containing two globular domains composed of beta sheets and alpha helices which form something like an inverse alpha/beta barrel. The two domains are connected by two strands which act like a hinge to bring the upper and lower domains together, sandwiching the substrates in the active site. Ligand binding converts the enzyme from an open state to a tightly-packed closed state, following the pattern of an induced-fit mechanism (Schönbrunn et al. (2001) Proc. Natl. Acad. Sci. USA 90:1376-1380, Stauffer et al. (2001) Biochemistry 40:3951-3957).

EPSP synthase has been isolated from plants, bacteria and fungi, including E. coli (Duncan et al. (1984) FEBS Lett. 170:59-63), Staphylococcus aureus (Horsburgh et al. (1996)Microbiology 142(Part 10):2943-2950), Streptococcus pneumoniae (Du et al. (2000) Eur. J. Biochem. 267(1):222-227) and Salmonella typhi (Chatfield et al. (1990) Nucleic Acids Res. 18(20):6133). Variants of the wild-type EPSP synthase enzyme have been isolated which are glyphosate tolerant as a result of alterations in the EPSP synthase amino acid coding sequence (Kishore and Shah (1988) Annu. Rev. Biochem. 57:627-63; Wang et al. (2003) J. Plant Res. 116:455-60; Eschenburg et al. (2002) Planta 216:129-35).

EPSP synthase sequences have been characterized and residues frequently conserved in this class of polypeptides have been identified. For example, Lys-22, Arg-124, Asp-313, Arg-344, Arg-386, and Lys-41 1, are conserved residues of the EPSP synthase from E. coli (Schönbrunn et al. (2001) Proc. Natl. Acad. Sci. USA 98:1376-1380). Additional residues that influence EPSP synthase activity also include Arg-100, Asp-242, and Asp-384 (Selvapandiyan et al. (1995) FEBS Letters 374:253-256). Arg-27 has been shown to bind to S3P (Shuttleworth et al. (1999) Biochemistry 38:296-302).

B. Glyphosate-Resistant EPSP Synthase

EPSP synthase is the target of the broad-spectrum herbicide glyphosate. By “glyphosate” is intended any herbicidal form of N-phosphonomethylglycine (including any salt thereof) and active derivatives thereof that result in the production of the glyphosate anion. Inhibition of EPSP synthase by glyphosate has been shown to proceed through the formation of an EPSP synthase-S3P-glyphosate ternary complex and the binding is ordered with glyphosate binding to the enzyme only after the formation of a binary EPSP synthase-S3P complex. Binding of glyphosate to EPSP synthase has been shown to be competitive with PEP and noncompetitive with respect to S3P (Kishore et al. (1988) Annu. Rev. Biochem. 57:627-663). By binding to EPSP synthase, glyphosate shuts down the shikimic acid pathway, thereby leading to a depletion of aromatic amino acid biosynthesis and death or severe growth reduction of the plant.

Glyphosate-resistant EPSP synthase polypeptides have been identified and used to increase glyphosate tolerance in plants. A “glyphosate resistance polypeptide” or “glyphosate tolerance polypeptide” includes a polypeptide that confers upon a cell the ability to tolerate a higher concentration of glyphosate than cells that do not express the polypeptide, or to tolerate a certain concentration of glyphosate for a longer period of time than cells that do not express the polypeptide. By “tolerate” or “tolerance” is intended either to survive, or to carry out essential cellular functions such as protein synthesis and respiration in a manner that is not readily discernable from untreated cells. An example of a naturally-occurring glyphosate-resistant EPSP synthase includes the bacterial gene from Agrobacterium tumefacians strain CP4 which has been used to confer herbicide resistance on plant cells following expression in plants. Mutated EPSP synthase polypeptides have been identified through random mutagenesis and selection for herbicide resistance, including a mutated EPSP synthase from Salmonella typhimurium strain CT7 that confers glyphosate resistance in bacterial cells, and confers glyphosate resistance on plant cells (U.S. Pat. Nos. 4,535,060; 4,769,061; and 5,094,945 and U.S. Appl. Nos. 60/669,686 and 20040177399). These enzymes contain amino acid substitutions in their active sites that prevent the binding of glyphosate without affecting binding by PEP or S3P. Mutations that occur in the hinge region between the two globular domains of EPSP synthase have been shown to alter the binding affinity of glyphosate but not PEP (He et al. (2003) Biosci. Biotechnol. Biochem. 67(6):1405-1409). Therefore, such enzymes have high catalytic activity, even in the presence of glyphosate.

EPSP synthase enzymes of the present invention are characterized as having a Q-loop region with increased polarity. Additionally, the enzymes may be characterized by having at least one domain selected from the domains listed below:
D-C-X₁-X₂-S-G (SEQ ID NO:29), where X₁ denotes glycine, serine, alanine or asparagine, and X₂ denotes asparagine or glutamic acid; or,
D-A-X₁-X₂-S-G (SEQ ID NO:30), where X₁ denotes alanine or arginine, and X₂ denotes asparagine or glutamic acid; or,
K-L-K-X₁-S-A (SEQ ID NO:31), where X₁ denotes glycine, asparagine or glutamic acid; or,
W-C-E-D-A-G (SEQ ID NO:32).

C. Activity of EPSP Synthase

A variety of methods can be used to measure EPSP synthase activity. For example, Lewendon et al. ((1983) Biochem J. 213:187-191) describes two assays which couple the EPSP synthase reaction with other enzymes which produced detectable products. In the forward direction, EPSP synthase can be coupled with chorismate synthase, the enzyme in the shikimate acid pathway that converts EPSP to chorismate; as EPSP synthase produces EPSP, chorismate synthase can convert EPSP to chorismate which can be detected at 275 nm. Since EPSP synthase can also proceed in the reverse direction, activity can also be assayed with coupling to pyruvate kinase and lactate dehydrogenase which oxidize NADH in the breakdown of pyruvate, allowing the detection of NADH loss at 340 nm which corresponds to pyruvate evolution by EPSP synthase. EPSP synthase activity can also be assayed by measuring an increase in resistance of a plant to glyphosate when glyphosate-resistant EPSP synthase is present, or by measuring an increase in plant yield when glyphosate-sensitive and/or -tolerant EPSP synthase is expressed.

D. Isolated Polynucleotides, and Variants and Fragments Thereof

In some embodiments, the present invention comprises isolated or purified polynucleotides other than the polynucleotides of SEQ ID NO:1, 3, 5, 11, 13, 38 or 40 (or any other known or published polynucleotide sequence encoding a polypeptide comprising one or more of the domains of the invention, for example SEQ ID NO:46-52) encoding polypeptides having a Q-loop region with increased polarity. Further embodiments include polynucleotides encoding polypeptides comprising one or more of the domains described above. An “isolated” or “purified” polynucleotide or polypeptide, or biologically active portion thereof, is substantially free of other cellular material, or culture medium when produced by recombinant techniques, or substantially free of chemical precursors or other chemicals when chemically synthesized. By “biologically active” is intended to possess the desired biological activity of the native polypeptide, that is, retain herbicide resistance or tolerance activity. An “isolated” polynucleotide may be free of sequences (for example, protein encoding sequences) that naturally flank the nucleic acid (i.e., sequences located at the 5′ and 3′ ends of the nucleic acid) in the genomic DNA of the organism from which the polynucleotide is derived. For purposes of the invention, “isolated” when used to refer to polynucleotides excludes isolated chromosomes. For example, in various embodiments, the isolated glyphosate resistance-encoding polynucleotide can contain less than about 5 kb, 4 kb, 3 kb, 2 kb, 1 kb, 0.5 kb, or 0.1 kb of nucleotide sequence that naturally flanks the polynucleotide in genomic DNA of the cell from which the polynucleotide is derived.

Polynucleotides of the invention include those encoding polypeptides characterized by having a Q-loop with increased polarity or at least one domain of the invention. The information used in identifying these domains includes sequence alignments of EPSP synthase enzymes as described elsewhere herein. The sequence alignments are used to identify regions of homology between the sequences and to identify the domains that are characteristic of these EPSP synthase enzymes. In some embodiments, the domains of the invention are used to identify EPSP synthase enzymes that are glyphosate resistant.

The present invention further contemplates variants and fragments of the polynucleotides described herein. A “fragment” of a polynucleotide may encode a biologically active portion of a polypeptide, or it may be a fragment that can be used as a hybridization probe or PCR primer using methods disclosed elsewhere herein. Polynucleotides that are fragments of a polynucleotide comprise at least about 15, 20, 50, 75, 100, 200, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950 contiguous nucleotides, or up to the number of nucleotides present in a full-length polynucleotide disclosed herein depending upon the intended use. By “contiguous” nucleotides is intended nucleotide residues that are immediately adjacent to one another.

Fragments of the polynucleotides of the present invention generally will encode polypeptide fragments that retain the biological activity of the full-length glyphosate resistance protein; i.e., herbicide-resistance activity. By “retains herbicide resistance activity” is intended that the fragment will have at least about 30%, at least about 50%, at least about 70%, or at least about 80% of the herbicide resistance activity of the full-length glyphosate resistance protein disclosed herein as SEQ ID NO:1. Methods for measuring herbicide resistance activity are well known in the art. See, for example, U.S. Pat. Nos. 4,535,060, and 5,188,642, each of which are herein incorporated by reference in their entirety.

A fragment of a polynucleotide that encodes a biologically active portion of a polypeptide of the invention will encode at least about 15, 25, 30, 50, 75, 100, 125, 150, 175, 200, 250, 300, 350, 400 contiguous amino acids, or up to the total number of amino acids present in a full-length polypeptide of the invention.

The invention also encompasses variant polynucleotides. “Variants” of the polynucleotide include those sequences that encode the polypeptides disclosed herein but that differ conservatively because of the degeneracy of the genetic code, as well as those that are sufficiently identical. The term “sufficiently identical” is intended a polypeptide or polynucleotide sequence that has at least about 60% or 65% sequence identity, about 70% or 75% sequence identity, about 80% or 85% sequence identity, about 90%, 91%, 92%, 93%, 94%, 95%, 96%, 97%, 98% or 99% sequence identity compared to a reference sequence using one of the alignment programs using standard parameters. One of skill in the art will recognize that these values can be appropriately adjusted to determine corresponding identity of polypeptides encoded by two polynucleotides by taking into account codon degeneracy, amino acid similarity, reading frame positioning, and the like.

To determine the percent identity of two amino acid sequences or of two polynucleotides, the sequences are aligned for optimal comparison purposes. The percent identity between the two sequences is a function of the number of identical positions shared by the sequences (i.e., percent identity=number of identical positions/total number of positions (e.g., overlapping positions)×100). In one embodiment, the two sequences are the same length. The percent identity between two sequences can be determined using techniques similar to those described below, with or without allowing gaps. In calculating percent identity, typically exact matches are counted.

The determination of percent identity between two sequences can be accomplished using a mathematical algorithm. A nonlimiting example of a mathematical algorithm utilized for the comparison of two sequences is the algorithm of Karlin and Altschul (1990) Proc. Natl. Acad. Sci. USA 87:2264, modified as in Karlin and Altschul (1993) Proc. Natl. Acad. Sci. USA 90:5873-5877. Such an algorithm is incorporated into the BLASTN and BLASTX programs of Altschul et al. (1990) J. Mol. Biol. 215:403. BLAST nucleotide searches can be performed with the BLASTN program, score=100, wordlength=12, to obtain polynucleotides homologous to herbicide resistance-encoding polynucleotides used in methods of the invention. BLAST polypeptide searches can be performed with the BLASTX program, score=50, wordlength=3, to obtain amino acid sequences homologous to polypeptide molecules expressed using the methods of the invention. To obtain gapped alignments for comparison purposes, Gapped BLAST can be utilized as described in Altschul et al. (1997) Nucleic Acids Res. 25:3389. Alternatively, PSI-Blast can be used to perform an iterated search that detects distant relationships between molecules. See Altschul et al. (1997) supra. When utilizing BLAST, Gapped BLAST, and PSI-Blast programs, the default parameters of the respective programs (e.g., BLASTX and BLASTN) can be used. See www.ncbi.nlm.nih.gov. Another non-limiting example of a mathematical algorithm utilized for the comparison of sequences is the ClustalW algorithm (Higgins et al. (1994) Nucleic Acids Res. 22:4673-4680). ClustalW compares sequences and aligns the entirety of the amino acid or DNA sequence, and thus can provide data about the sequence conservation of the entire amino acid sequence. The ClustalW algorithm is used in several commercially available DNA/amino acid analysis software packages, such as the ALIGNX module of the Vector NTI Program Suite (Invitrogen Corporation, Carlsbad, Calif.). After alignment of amino acid sequences with ClustalW, the percent amino acid identity can be assessed. A non-limiting example of a software program useful for analysis of ClustalW alignments is GeneDoc™. Genedoc™ (Karl Nicholas) allows assessment of amino acid (or DNA) similarity and identity between multiple polypeptides. Another non-limiting example of a mathematical algorithm utilized for the comparison of sequences is the algorithm of Myers and Miller (1988) CABIOS 4:11-17. Such an algorithm is incorporated into the ALIGN program (version 2.0), which is part of the GCG sequence alignment software package. When utilizing the ALIGN program for comparing amino acid sequences, a PAM 120 weight residue table, a gap length penalty of 12, and a gap penalty of 4 can be used.

Unless otherwise stated, GAP Version 10, which uses the algorithm of Needleman and Wunsch (1970) supra, will be used to determine sequence identity or similarity using the following parameters: % identity and % similarity for a nucleotide sequence using GAP Weight of 50 and Length Weight of 3, and the nwsgapdna.cmp scoring matrix; % identity or % similarity for an amino acid sequence using GAP weight of 8 and length weight of 2, and the BLOSUM62 scoring program. Equivalent programs may also be used. By “equivalent program” is intended any sequence comparison program that, for any two sequences in question, generates an alignment having identical nucleotide residue matches and an identical percent sequence identity when compared to the corresponding alignment generated by GAP Version 10.

Naturally occurring allelic variants can be identified with the use of well-known molecular biology techniques, such as polymerase chain reaction (PCR) and hybridization techniques as outlined below. Variant polynucleotides also include synthetically derived polynucleotides that have been generated, for example, by using site-directed mutagenesis but which still encode the polypeptide having the desired biological activity.

The skilled artisan will further appreciate that changes can be introduced by mutation into the polynucleotides of the invention thereby leading to changes in the amino acid sequence of the encoded polypeptides, without altering the biological activity of the polypeptides. Thus, variant isolated polynucleotides can be created by introducing one or more nucleotide substitutions, additions, or deletions into the corresponding polynucleotide disclosed herein, such that one or more amino acid substitutions, additions or deletions are introduced into the encoded polypeptide. Mutations can be introduced by standard techniques, such as site-directed mutagenesis and PCR-mediated mutagenesis, or gene shuffling techniques. Such variant polynucleotides are also encompassed by the present invention.

Variant polynucleotides can be made by introducing mutations randomly along all or part of the coding sequence, such as by saturation mutagenesis, and the resultant mutants can be screened for the ability to confer herbicide resistance activity to identify mutants that retain activity. Following mutagenesis, the encoded polypeptide can be expressed recombinantly, and the activity of the polypeptide can be determined using standard assay techniques.

Gene shuffling or sexual PCR procedures (for example, Smith (1994) Nature 370:324-325; U.S. Pat. Nos. 5,837,458; 5,830,721; 5,811,238; and 5,733,731, each of which is herein incorporated by reference) can be used to identify additional polynucleotides that encode polypeptides that perform similar functions as those described herein (for example, polypeptides that confer glyphosate resistance). Gene shuffling involves random fragmentation of several mutant DNAs followed by their reassembly by PCR into full length molecules. Examples of various gene shuffling procedures include, but are not limited to, assembly following DNase treatment, the staggered extension process (STEP), and random priming in vitro recombination. In the DNase mediated method, DNA segments isolated from a pool of positive mutants are cleaved into random fragments with DNaseI and subjected to multiple rounds of PCR with no added primer. The lengths of random fragments approach that of the uncleaved segment as the PCR cycles proceed, resulting in mutations in different clones becoming mixed and accumulating in some of the resulting sequences. Multiple cycles of selection and shuffling have led to the functional enhancement of several enzymes (Stemmer (1994) Nature 370:389-391; Stemmer (1994) Proc. Natl. Acad. Sci. USA 91:10747-10751; Crameri et al. (1996) Nat. Biotechnol. 14:315-319; Zhang et al. (1997) Proc. Natl. Acad. Sci. USA 94:4504-4509; and Crameri et al. (1997) Nat. Biotechnol. 15:436-438). Such procedures could be performed, for example, on polynucleotides encoding EPSP synthase enzymes having a Q-loop region with increased polarity or polypeptides comprising domains of the present invention to generate polypeptides that confer glyphosate resistance.

Using methods such as PCR, hybridization, and the like corresponding herbicide resistance sequences can be identified by looking for the conserved domains of the invention. See, for example, Sambrook and Russell (2001) Molecular Cloning: A Laboratory Manual (Cold Spring Harbor Laboratory Press, Cold Spring Harbor, N.Y.) and Innis et al. (1990) PCR Protocols: A Guide to Methods and Applications (Academic Press, NY).

In a hybridization method, all or part of the herbicide resistance polynucleotide sequence or a sequence encoding a domain of the invention can be used to screen cDNA or genomic libraries. Methods for construction of such cDNA and genomic libraries are generally known in the art and are disclosed in Sambrook and Russell, 2001, supra. The so-called hybridization probes may be genomic DNA fragments, cDNA fragments, RNA fragments, or other oligonucleotides, and may be labeled with a detectable group such as ³²P, or any other detectable marker, such as other radioisotopes, a fluorescent compound, an enzyme, or an enzyme co-factor. Probes for hybridization can be made by labeling synthetic oligonucleotides based on the known herbicide resistance-encoding nucleotide sequence disclosed herein. Degenerate primers designed on the basis of conserved nucleotides or amino acid residues in the nucleotide sequence or encoded amino acid sequence can additionally be used. The probe typically comprises a region of nucleotide sequence that hybridizes under stringent conditions to at least about 12, at least about 25, at least about 50, 75, 100, 125, 150, 175, 200, 250, 300, 350, 400, 500, 600, 700, 800, 900, 1000, 1200, 1400, 1600, or 1800 consecutive nucleotides of the herbicide resistance-encoding polynucleotide of the invention or a fragment or variant thereof. Methods for the preparation of probes for hybridization are generally known in the art and are disclosed in Sambrook and Russell (2001) supra, and Sambrook et al. (1989) Molecular Cloning. A Laboratory Manual (2d ed., Cold Spring Harbor Laboratory Press, Cold Spring Harbor, N.Y.), both of which are herein incorporated by reference.

Hybridization of such sequences may be carried out under stringent conditions. By “stringent conditions” or “stringent hybridization conditions” is intended conditions under which a probe will hybridize to its target sequence to a detectably greater degree than to other sequences (e.g., at least 2-fold over background). Stringent conditions are sequence-dependent and will be different in different circumstances. By controlling the stringency of the hybridization and/or washing conditions, target sequences that are 100% complementary to the probe can be identified (homologous probing). Alternatively, stringency conditions can be adjusted to allow some mismatching in sequences so that lower degrees of similarity are detected (heterologous probing). Generally, a probe is less than about 1000 nucleotides in length, or less than about 500 nucleotides in length.

Stringent conditions will be those in which the salt concentration is less than about 1.5 M Na ion, or about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulfate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C. Optionally, wash buffers may comprise about 0.1% to about 1% SDS. Duration of hybridization is generally less than about 24 hours, usually about 4 to about 12 hours.

Specificity is typically the function of post-hybridization washes, the critical factors being the ionic strength and temperature of the final wash solution. For DNA-DNA hybrids, the Tₘ can be approximated from the equation of Meinkoth and Wahl (1984) Anal. Biochem. 138:267-284: Tₘ=81.5° C.+16.6 (log M)+0.41 (% GC)−0.61 (% form)−500/L; where M is the molarity of monovalent cations, % GC is the percentage of guanosine and cytosine nucleotides in the polynucleotide sequence, % form is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Tₘ is reduced by about 1° C. for each 1% of mismatching; thus, Tₘ, hybridization, and/or wash conditions can be adjusted to hybridize to sequences of the desired identity. For example, if sequences with ≧90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3, or 4° C. lower than the thermal melting point (Tₘ); moderately stringent conditions can utilize a hybridization and/or wash at 6, 7, 8, 9, or 10° C. lower than the thermal melting point (Tₘ); low stringency conditions can utilize a hybridization and/or wash at 11, 12, 13, 14, 15, or 20° C. lower than the thermal melting point (Tₘ). Using the equation, hybridization and wash conditions, and desired Tₘ, those of ordinary skill in the art will understand that variations in the stringency of hybridization and/or wash solutions are inherently described. If the desired degree of mismatching results in a Tₘ of less than 45° C. (aqueous solution) or 32° C. (formamide solution), the SSC concentration can be increased so that a higher temperature can be used. An extensive guide to the hybridization of nucleic acids is found in Tijssen (1993) Laboratory Techniques in Biochemistry and Molecular Biolog-Hybridization with Nucleic Acid Probes, Part I, Chapter 2 (Elsevier, New York); and Ausubel et al, eds. (1995) Current Protocols in Molecular Biology, Chapter 2 (Greene Publishing and Wiley-Interscience, New York). See Sambrook et al. (1989) Molecular Cloning: A Laboratory Manual (2d ed., Cold Spring Harbor Laboratory Press, Cold Spring Harbor, N.Y.).

E. Isolated Proteins and Variants and Fragments Thereof

In some embodiments, the present invention comprises isolated or purified herbicide resistance polypeptides other than SEQ ID NO:2, 4, 7, 12, 14, 39, and 41 (or any other known or published polypeptide comprising one or more of the domains of the invention, for example SEQ ID NO:46-52). An “isolated” or “purified” herbicide resistance polypeptide that is substantially free of cellular material includes preparations of polypeptides having less than about 30%, 20%, 10%, or 5% (by dry weight) of non-herbicide resistance polypeptide (also referred to herein as a “contaminating protein”). In the present invention, “herbicide resistance protein” is intended an EPSP synthase enzyme having a Q-loop region with increased polarity, or having at least one of the domains of the invention. Fragments, biologically active portions, and variants thereof are also provided, and may be used to practice the methods of the present invention.

“Fragments” or “biologically active portions” include polypeptide fragments comprising a portion of an amino acid sequence encoding an herbicide resistance protein and that retains herbicide resistance activity. A biologically active portion of an herbicide resistance protein can be a polypeptide that is, for example, 10, 25, 50, 100 or more amino acids in length. Such biologically active portions can be prepared by recombinant techniques and evaluated for herbicide resistance activity.

By “variants” is intended proteins or polypeptides having an amino acid sequence that is at least about 60%, 65%, about 70%, 75%, about 80%, 85%, 90%, 91%, 92%, 93%, 94%, 95%, 96%, 97%, 98% or 99% identical to an EPSP synthase polypeptide having a Q-loop region with increased polarity, or an EPSP synthase polypeptide having a domain of the present invention. One of skill in the art will recognize that these values can be appropriately adjusted to determine corresponding identity of polypeptides encoded by two polynucleotides by taking into account codon degeneracy, amino acid similarity, reading frame positioning, and the like.

For example, conservative amino acid substitutions may be made at one or more nonessential amino acid residues. A “nonessential” amino acid residue is a residue that can be altered from the wild-type sequence of a polypeptide without substantially altering the biological activity of the resulting peptide, whereas an “essential” amino acid residue a residue that cannot be substituted without substantially affecting biological activity. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Amino acid substitutions may be made in nonconserved regions that retain function. In general, such substitutions would not be made for conserved amino acid residues, or for amino acid residues residing within a conserved motif, where such residues are essential for polypeptide activity. However, one of skill in the art would understand that functional variants may have minor conserved or nonconserved alterations in the conserved residues.

Amino acid substitutions that are made to increase the polarity and/or bulkiness of the EPSP synthase binding pocket for PEP and glyphosate (herein referred to as the “Q-loop”) are also encompassed by the present invention. This loop forms a portion of the binding pocket for PEP and glyphosate, and contains an invariant arginine that is known to hydrogen bond directly with the phosphate of PEP (Shuttleworth et al. (1999) Biochemistry 38:296-302). For the purposes of the present invention, an increase in the polarity of this region refers to an increase in the number or relative percent composition of polar and/or charged amino acids in a given polypeptide sequence relative to the polypeptide sequence in this region of E. coli AroA (SEQ ID NO:22), which is an example of an EPSP synthase enzyme not having a domain of the present invention. For example, the substitution of an aspartic acid residue for a phenylalanine residue at position 1 of SEQ ID NO:33 and 34 (which corresponds to an example sequence in the Q-loop region) may, while not being bound by any mechanism of action, result in charge repulsion between the loop and the negatively charged phosphonate residue of glyphosate. Methods and algorithms for estimating the net charge and/or net polarity of a particular amino acid composition are known in the art.

An increase in bulk (for example, by the substitution of the more bulky lysine residues at positions 1 and 3, respectively, of SEQ ID NO:31, in place of the less bulky phenylalanine and glycine residues present in other EPSP synthases) in this loop may, while not bound by any mechanism of action, result in steric effects resulting in a downward displacement of this loop further into the binding pocket, reducing the size of the active site pocket. Polypeptides (as well as the polynucleotides encoding them) in which an increase in bulk in the Q-loop has been introduced by substitution of one or more residues in the Q-loop for a more bulky residue are also encompassed by the present invention.

In another embodiment of the present invention, the domains identified herein may be engineered or recombined with the amino acid sequences of other enzymes, for example, by replacement of the Class I EPSP synthase motif of the E. coli aroA gene with a polypeptide having a Q-loop with increased polarity or with a polypeptide comprising a domain of the present invention. Alternatively, one or more of these polypeptide(s) may be inserted in replace of a polypeptide that does not comprise a Q-loop region with increased polarity or with a polypeptide comprising a domain of the invention, which may or may not comprise or result in improved properties.

Variants also include polypeptides encoded by a polynucleotide that hybridizes to the polynucleotide encoding an enzyme having a Q-loop region with increased polarity or a domain of the present invention, or a complement thereof, under stringent conditions. Variants include polypeptides that differ in amino acid sequence due to mutagenesis. Variant proteins encompassed by the present invention are biologically active, that is they continue to possess the desired biological activity of the native protein, that is, retain herbicide resistance activity. Methods for measuring herbicide resistance activity are well known in the art. See, for example, U.S. Pat. Nos. 4,535,060, and 5,188,642, each of which are herein incorporated by reference in their entirety.

Bacterial genes quite often possess multiple methionine initiation codons in proximity to the start of the open reading frame. Often, translation initiation at one or more of these start codons will lead to generation of a functional protein. These start codons can include ATG codons. However, bacteria such as Bacillus sp. also recognize the codon GTG as a start codon, and proteins that initiate translation at GTG codons contain a methionine at the first amino acid. Furthermore, it is not often determined a priori which of these codons are used naturally in the bacterium. Thus, it is understood that use of one of the alternate methionine codons may lead to generation of variants that confer herbicide resistance. These herbicide resistance proteins are encompassed in the present invention and may be used in the methods of the present invention.

Antibodies to the polypeptides of the present invention, or to variants or fragments thereof, are also encompassed. Methods for producing antibodies are well known in the art (see, for example, Harlow and Lane (1988) Antibodies: A Laboratory Manual, Cold Spring Harbor Laboratory, Cold Spring Harbor, N.Y.; U.S. Pat. No. 4,196,265).

F. Polynucleotide Constructs

The polynucleotides employed in the methods and compositions of the invention may be modified to obtain or enhance expression in plant cells. The polynucleotides encoding the domains of the invention may be provided in expression cassettes for expression in the plant of interest. A “plant expression cassette” includes a DNA construct that is capable of resulting in the expression of a polynucleotide in a plant cell. The cassette can include in the 5′-3′ direction of transcription, a transcriptional initiation region (i.e., promoter) operably-linked to one or more polynucleotides of interest, and a translation and transcriptional termination region (i.e., termination region) functional in plants. The cassette may additionally contain at least one additional polynucleotide to be introduced into the organism, such as a selectable marker gene. Alternatively, the additional polynucleotide(s) can be provided on multiple expression cassettes. Such an expression cassette is provided with a plurality of restriction sites for insertion of the polynucleotide(s) to be under the transcriptional regulation of the regulatory regions.

“Heterologous” generally refers to the polynucleotide or polypeptide that is not endogenous to the cell or is not endogenous to the location in the native genome in which it is present, and has been added to the cell by infection, transfection, microinjection, electroporation, microprojection, or the like. By “operably linked” is intended a functional linkage between two polynucleotides. For example, when a promoter is operably linked to a DNA sequence, the promoter sequence initiates and mediates transcription of the DNA sequence. It is recognized that operably linked polynucleotides may or may not be contiguous and, where used to reference the joining of two polypeptide coding regions, the polypeptides are expressed in the same reading frame.

The promoter may be any polynucleotide sequence which shows transcriptional activity in the chosen plant cells, plant parts, or plants. The promoter may be native or homologous, or foreign or heterologous, to the plant host and/or to the DNA sequence of the invention. Where the promoter is “native” or “homologous” to the plant host, it is intended that the promoter is found in the native plant into which the promoter is introduced. Where the promoter is “foreign” or “heterologous” to the DNA sequence of the invention, it is intended that the promoter is not the native or naturally occurring promoter for the operably linked DNA sequence of the invention. The promoter may be inducible or constitutive. It may be naturally-occurring, may be composed of portions of various naturally-occurring promoters, or may be partially or totally synthetic. Guidance for the design of promoters is provided by studies of promoter structure, such as that of Harley and Reynolds (1987) Nucleic Acids Res. 15:2343-2361. Also, the location of the promoter relative to the transcription start may be optimized. See, e.g., Roberts et al. (1979) Proc. Natl. Acad. Sci. USA, 76:760-764. Many suitable promoters for use in plants are well known in the art.

For instance, suitable constitutive promoters for use in plants include: the promoters from plant viruses, such as the peanut chlorotic streak caulimovirus (PCISV) promoter (U.S. Pat. No. 5,850,019); the 35S promoter from cauliflower mosaic virus (CaMV) (Odell et al. (1985) Nature 313:810-812); promoters of Chlorella virus methyltransferase genes (U.S. Pat. No. 5,563,328) and the full-length transcript promoter from figwort mosaic virus (FMV) (U.S. Pat. No. 5,378,619); the promoters from such genes as rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten et al. (1984) EMBO J. 3:2723-2730); maize H3 histone (Lepetit et al. (1992) Mol. Gen. Genet. 231:276-285 and Atanassova et al. (1992) Plant J. 2(3):291-300); Brassica napus ALS3 (PCT application WO 97/41228); and promoters of various Agrobacterium genes (see U.S. Pat. Nos. 4,771,002; 5,102,796; 5,182,200; and 5,428,147).

Suitable inducible promoters for use in plants include: the promoter from the ACEI system which responds to copper (Mett et al. (1993) PNAS 90:4567-4571); the promoter of the maize In2 gene which responds to benzenesulfonamide herbicide safeners (Hershey et al. (1991) Mol. Gen. Genetics 227:229-237 and Gatz et al. (1994) Mol. Gen. Genetics 243:32-38); and the promoter of the Tet repressor from Tn10 (Gatz et al. (1991) Mol. Gen. Genet. 227:229-237). Another inducible promoter for use in plants is one that responds to an inducing agent to which plants do not normally respond. An exemplary inducible promoter of this type is the inducible promoter from a steroid hormone gene, the transcriptional activity of which is induced by a glucocorticosteroid hormone (Schena et al. (1991) Proc. Natl. Acad. Sci. USA 88:10421) or the recent application of a chimeric transcription activator, XVE, for use in an estrogen receptor-based inducible plant expression system activated by estradiol (Zuo et al. (2000) Plant J., 24:265-273). Other inducible promoters for use in plants are described in EP 332104, PCT WO 93/21334 and PCT WO 97/06269 which are herein incorporated by reference in their entirety. Promoters composed of portions of other promoters and partially or totally synthetic promoters can also be used. See, e.g., Ni et al. (1995) Plant J. 7:661-676 and PCT WO 95/14098 describing such promoters for use in plants.

The promoter may include, or be modified to include, one or more enhancer elements. In some embodiments, the promoter may include a plurality of enhancer elements. Promoters containing enhancer elements provide for higher levels of transcription as compared to promoters that do not include them. Suitable enhancer elements for use in plants include the PCISV enhancer element (U.S. Pat. No. 5,850,019), the CaMV 35S enhancer element (U.S. Pat. Nos. 5,106,739 and 5,164,316) and the FMV enhancer element (Maiti et al. (1997) Transgenic Res. 6:143-156). See also PCT WO 96/23898.

Often, such constructs can contain 5′ and 3′ untranslated regions. Such constructs may contain a “signal sequence” or “leader sequence” to facilitate co-translational or post-translational transport of the peptide of interest to certain intracellular structures such as the chloroplast (or other plastid), endoplasmic reticulum, or Golgi apparatus, or to be secreted. For example, the construct can be engineered to contain a signal peptide to facilitate transfer of the peptide to the endoplasmic reticulum. By “signal sequence” is intended a sequence that is known or suspected to result in cotranslational or post-translational peptide transport across the cell membrane. In eukaryotes, this typically involves secretion into the Golgi apparatus, with some resulting glycosylation. By “leader sequence” is intended any sequence that, when translated, results in an amino acid sequence sufficient to trigger co-translational transport of the peptide chain to a sub-cellular organelle. Thus, this includes leader sequences targeting transport and/or glycosylation by passage into the endoplasmic reticulum, passage to vacuoles, plastids including chloroplasts, mitochondria, and the like. It may also be preferable to engineer the plant expression cassette to contain an intron, such that mRNA processing of the intron is required for expression.

By “3′ untranslated region” is intended a polynucleotide located downstream of a coding sequence. Polyadenylation signal sequences and other sequences encoding regulatory signals capable of affecting the addition of polyadenylic acid tracts to the 3′ end of the mRNA precursor are 3′ untranslated regions. By “5′ untranslated region” is intended a polynucleotide located upstream of a coding sequence.

Other upstream or downstream untranslated elements include enhancers. Enhancers are polynucleotides that act to increase the expression of a promoter region. Enhancers are well known in the art and include, but are not limited to, the SV40 enhancer region and the 35S enhancer element.

The termination region may be native with the transcriptional initiation region, may be native with the sequence of the present invention, or may be derived from another source. Convenient termination regions are available from the Ti-plasmid of A. tumefaciens, such as the octopine synthase and nopaline synthase termination regions. See also Guerineau et al. (1991) Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. (1989) Nucleic Acids Res. 17:7891-7903; and Joshi et al. (1987) Nucleic Acid Res. 15:9627-9639.

Where appropriate, the polynucleotide(s) encoding the polypeptide domains of the invention may be optimized for increased expression in the transformed host cell. That is, the sequences can be synthesized using host cell-preferred codons for improved expression, or may be synthesized using codons at a host-preferred codon usage frequency. Generally, the GC content of the polynucleotide will be increased. See, for example, Campbell and Gowri (1990) Plant Physiol. 92: 1-11 for a discussion of host-preferred codon usage. Methods are known in the art for synthesizing host-preferred polynucleotides. See, for example, U.S. Pat. Nos. 6,320,100; 6,075,185; 5,380,831; and 5,436,391, U.S. Published Application Nos. 20040005600 and 20010003849, and Murray et al. (1989) Nucleic Acids Res. 17:477-498, herein incorporated by reference.

In one embodiment, the polynucleotides of interest are targeted to the chloroplast for expression. In this manner, where the polynucleotide of interest is not directly inserted into the chloroplast, the expression cassette will additionally contain a polynucleotide encoding a transit peptide to direct the nucleotide of interest to the chloroplasts. Such transit peptides are known in the art. See, for example, Von Heijne et al. (1991) Plant Mol. Biol. Rep. 9:104-126; Clark et al. (1989) J. Biol. Chem. 264:17544-17550; Della-Cioppa et al. (1987) Plant Physiol. 84:965-968; Romer et al. (1993) Biochem. Biophys. Res. Commun. 196:1414-1421; and Shah et al. (1986) Science 233:478-481.

The polynucleotides of interest to be targeted to the chloroplast may be optimized for expression in the chloroplast to account for differences in codon usage between the plant nucleus and this organelle. In this manner, the polynucleotides of interest may be synthesized using chloroplast-preferred codons. See, for example, U.S. Pat. No. 5,380,831, herein incorporated by reference.

This plant expression cassette can be inserted into a plant transformation vector. By “transformation vector” is intended a DNA molecule that allows for the transformation of a cell. Such a molecule may consist of one or more expression cassettes, and may be organized into more than one vector DNA molecule. For example, binary vectors are plant transformation vectors that utilize two non-contiguous DNA vectors to encode all requisite cis- and trans-acting functions for transformation of plant cells (Hellens and Mullineaux (2000) Trends in Plant Science 5:446-451). “Vector” refers to a polynucleotide construct designed for transfer between different host cells. “Expression vector” refers to a vector that has the ability to incorporate, integrate and express heterologous DNA sequences or fragments in a foreign cell.

The plant transformation vector comprises one or more DNA vectors for achieving plant transformation. For example, it is a common practice in the art to utilize plant transformation vectors that comprise more than one contiguous DNA segment. These vectors are often referred to in the art as binary vectors. Binary vectors as well as vectors with helper plasmids are most often used for Agrobacterium-mediated transformation, where the size and complexity of DNA segments needed to achieve efficient transformation is quite large, and it is advantageous to separate functions onto separate DNA molecules. Binary vectors typically contain a plasmid vector that contains the cis-acting sequences required for T-DNA transfer (such as left border and right border), a selectable marker that is engineered to be capable of expression in a plant cell, and a “polynucleotide of interest” (a polynucleotide engineered to be capable of expression in a plant cell for which generation of transgenic plants is desired). Also present on this plasmid vector are sequences required for bacterial replication. The cis-acting sequences are arranged in a fashion to allow efficient transfer into plant cells and expression therein. For example, the selectable marker sequence and the sequence of interest are located between the left and right borders. Often a second plasmid vector contains the trans-acting factors that mediate T-DNA transfer from Agrobacterium to plant cells. This plasmid often contains the virulence functions (Vir genes) that allow infection of plant cells by Agrobacterium, and transfer of DNA by cleavage at border sequences and vir-mediated DNA transfer, as is understood in the art (Hellens and Mullineaux (2000) Trends in Plant Science, 5:446-451). Several types of Agrobacterium strains (e.g., LBA4404, GV3101, EHA101, EHA105, etc.) can be used for plant transformation. The second plasmid vector is not necessary for introduction of polynucleotides into plants by other methods such as microprojection, microinjection, electroporation, polyethylene glycol, etc.

G. Plants and Plant Parts

By “plant” is intended whole plants, plant organs (e.g., leaves, stems, roots, etc.), seeds, plant cells, propagules, embryos and progeny of the same. Plant cells can be differentiated or undifferentiated (e.g., callus, suspension culture cells, protoplasts, leaf cells, root cells, phloem cells, pollen). The present invention may be used for introduction of polynucleotides into any plant species, including, but not limited to, monocots and dicots. Examples of plants of interest include, but are not limited to, corn (maize), sorghum, wheat, sunflower, tomato, crucifers, peppers, potato, cotton, rice, soybean, sugarbeet, sugarcane, tobacco, barley, and oilseed rape, Brassica sp., alfalfa, rye, millet, safflower, peanuts, sweet potato, cassava, coffee, coconut, pineapple, citrus trees, cocoa, tea, banana, avocado, fig, guava, mango, olive, papaya, cashew, macadamia, almond, oats, vegetables, ornamentals, and conifers.

Vegetables include, but are not limited to, tomatoes, lettuce, green beans, lima beans, peas, and members of the genus Curcumis such as cucumber, cantaloupe, and musk melon. Ornamentals include, but are not limited to, azalea, hydrangea, hibiscus, roses, tulips, daffodils, petunias, carnation, poinsettia, and chrysanthemum. Crop plants are also of interest, including, for example, maize, sorghum, wheat, sunflower, tomato, crucifers, peppers, potato, cotton, rice, soybean, sugarbeet, sugarcane, tobacco, barley, oilseed rape, etc.

This invention is suitable for any member of the monocot plant family including, but not limited to, maize, rice, barley, oats, wheat, sorghum, rye, sugarcane, pineapple, yams, onion, banana, coconut, and dates.

II. Methods

A. Plant Transformation

Methods of the invention involve introducing one or more polynucleotides other than SEQ ID NO:1, 13 and 38 (or any other known or published polynucleotide sequence encoding a polypeptide comprising one or more of the domains of the invention, for example SEQ ID NO:46-52) into a plant. By “introducing” is intended to present to the plant the polynucleotide in such a manner that the polynucleotide gains access to the interior of a cell of the plant. The methods of the invention do not require that a particular method for introducing a polynucleotide into a plant be used, only that the polynucleotide gains access to the interior of at least one cell of the plant.

Introduction of a polynucleotide into plant cells is accomplished by one of several techniques known in the art, including but not limited to electroporation or chemical transformation (See, for example, Ausubel, ed. (1994) Current Protocols in Molecular Biology (John Wiley and Sons, Inc., Indianapolis, Ind.). Markers conferring resistance to toxic substances are useful in identifying transformed cells (having taken up and expressed the test polynucleotide sequence) from non-transformed cells (those not containing or not expressing the test polynucleotide sequence). In one aspect of the invention, genes are useful as a marker to assess introduction of DNA into plant cells. “Transgenic plants” or “transformed plants” or “stably transformed” plants, cells, tissues or seed refer to plants that have incorporated or integrated exogenous polynucleotides into the plant cell. By “stable transformation” is intended that the polynucleotide construct introduced into a plant integrates into the genome of the plant and is capable of being inherited by progeny thereof.

In general, plant transformation methods involve transferring heterologous DNA into target plant cells (e.g., immature or mature embryos, suspension cultures, undifferentiated callus, protoplasts, etc.), followed by applying a maximum threshold level of appropriate selection (depending on the selectable marker gene) to recover the transformed plant cells from a group of untransformed cell mass. Explants are typically transferred to a fresh supply of the same medium and cultured routinely. Subsequently, the transformed cells are differentiated into shoots after placing on regeneration medium supplemented with a maximum threshold level of selecting agent (i.e., temperature and/or herbicide). The shoots are then transferred to a selective rooting medium for recovering rooted shoot or plantlet. The transgenic plantlet then grow into mature plants and produce fertile seeds (e.g., Hiei et al. (1994) Plant J. 6:271-282; Ishida et al. (1996) Nat. Biotechnol. 14:745-750). A general description of the techniques and methods for generating transgenic plants is found in Ayres and Park (1994) CRC Crit. Rev. Plant Sci. 13:219-239 and Bommineni and Jauhar (1997) Maydica 42:107-120. Since the transformed material contains many cells, both transformed and non-transformed cells are present in any piece of subjected target callus or tissue or group of cells. The ability to kill non-transformed cells and allow transformed cells to proliferate results in transformed plant cultures. Often, the ability to remove non-transformed cells is a limitation to rapid recovery of transformed plant cells and successful generation of transgenic plants. Molecular and biochemical methods may be used to confirm the presence of the integrated polynucleotide(s) of interest in the genome of transgenic plant.

Generation of transgenic plants may be performed by one of several methods, including but not limited to introduction of heterologous DNA by Agrobacterium into plant cells (Agrobacterium-mediated transformation), bombardment of plant cells with heterologous foreign DNA adhered to particles, and various other non-particle direct-mediated methods (e.g., Hiei et al. (1994) Plant J. 6:271-282; Ishida et al. (1996) Nat. Biotechnol. 14:745-750; Ayres and Park (1994) CRC Crit. Rev. Plant Sci. 13:219-239; Bommineni and Jauhar (1997) Maydica 42:107-120) to transfer DNA.

There are three common methods of transforming plant cells with Agrobacterium. The first method is co-cultivation of Agrobacterium with cultured isolated protoplasts. This method requires an established culture system that allows culturing protoplasts and plant regeneration from cultured protoplasts. The second method is transformation of cells or tissues with Agrobacterium. This method requires (a) that the plant cells or tissues can be transformed by Agrobacterium and (b) that the transformed cells or tissues can be induced to regenerate into whole plants. The third method is transformation of seeds, apices or meristems with Agrobacterium. This method requires micropropagation.

The efficiency of transformation by Agrobacterium may be enhanced by using a number of methods known in the art. For example, the inclusion of a natural wound response molecule such as acetosyringone (AS) to the Agrobacterium culture has been shown to enhance transformation efficiency with Agrobacterium tumefaciens (Shahla et al. (1987) Plant Molec. Biol. 8:291-298). Alternatively, transformation efficiency may be enhanced by wounding the target tissue to be transformed. Wounding of plant tissue may be achieved, for example, by punching, maceration, bombardment with microprojectiles, etc. See, for example, Bidney et al. (1992) Plant Molec. Biol. 18:301-313.

In still further embodiments, the plant cells are transfected with vectors via particle bombardment (i.e., with a gene gun). Particle mediated gene transfer methods are known in the art, are commercially available, and include, but are not limited to, the gas driven gene delivery instrument described in U.S. Pat. No. 5,584,807, the entire contents of which are herein incorporated by reference. This method involves coating the polynucleotide sequence of interest onto heavy metal particles, and accelerating the coated particles under the pressure of compressed gas for delivery to the target tissue.

Other particle bombardment methods are also available for the introduction of heterologous polynucleotide sequences into plant cells. Generally, these methods involve depositing the polynucleotide sequence of interest upon the surface of small, dense particles of a material such as gold, platinum, or tungsten. The coated particles are themselves then coated onto either a rigid surface, such as a metal plate, or onto a carrier sheet made of a fragile material such as mylar. The coated sheet is then accelerated toward the target biological tissue. The use of the flat sheet generates a uniform spread of accelerated particles that maximizes the number of cells receiving particles under uniform conditions, resulting in the introduction of the polynucleotide sample into the target tissue.

Specific initiation signals may also be used to achieve more efficient translation of sequences encoding the polypeptide of interest. Such signals include the ATG initiation codon and adjacent sequences. In cases where sequences encoding the polypeptide of interest, its initiation codon, and upstream sequences are inserted into the appropriate expression vector, no additional transcriptional or translational control signals may be needed. However, in cases where only the coding sequence, or a portion thereof, is inserted, exogenous translational control signals including the ATG initiation codon should be provided. Furthermore, the initiation codon should be in the correct reading frame to ensure translation of the entire insert. Exogenous translational elements and initiation codons may be of various origins, both natural and synthetic. The efficiency of expression may be enhanced by the inclusion of enhancers that are appropriate for the particular cell system that is used, such as those described in the literature (Scharf et al. (1994) Results Probl. Cell Differ. 20:125).

Cells that have been transformed with a polynucleotide other than SEQ ID NO:1, 13, and 38 (or any other known or published polynucleotide sequence encoding a polypeptide comprising one or more of the domains of the invention, for example SEQ ID NO:46-52) encoding a polypeptide domain of the invention may be grown into plants in accordance with conventional ways. See, for example, McCormick et al. (1986) Plant Cell Rep. 5:81-84. These plants may then be grown, and pollinated with either the same transformed strain or different strains, and the resulting hybrid having constitutive expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. In this manner, the present invention provides transformed seed (also referred to as “transgenic seed”) having a polynucleotide encoding a polypeptide domain of the invention, for example, an expression cassette of the invention, stably incorporated into their genome.

B. Evaluation of Plant Transformation

Following introduction of DNA into plant cells, the transformation or integration of the polynucleotide into the plant genome is confirmed by various methods such as analysis of polynucleotides, polypeptides and metabolites associated with the integrated sequence.

PCR analysis is a rapid method to screen cells, tissue or shoots for the presence of incorporated gene at the earlier stage before transplanting into the soil (Sambrook and Russell (2001) Molecular Cloning: A Laboratory Manual (Cold Spring Harbor Laboratory Press, Cold Spring Harbor, N.Y.)). PCR is carried out using oligonucleotide primers specific to the nucleotide of interest or Agrobacterium vector background, etc.

Introduction of DNA may be confirmed by Southern blot analysis of genomic DNA (Sambrook and Russell (2001) supra). In general, total DNA is extracted from the cell or organism, digested with appropriate restriction enzymes, fractionated in an agarose gel and transferred to a nitrocellulose or nylon membrane. The membrane or “blot” is then probed with, for example, radiolabeled ³²P target DNA fragment to confirm the integration of introduced DNA into the plant genome according to standard techniques (Sambrook and Russell (2001) supra).

In Northern analysis, RNA is isolated from specific tissues of the cell or organism, fractionated in a formaldehyde agarose gel and blotted onto a nylon filter according to standard procedures that are routinely used in the art (Sambrook and Russell (2001) supra). Expression of RNA encoded by the polynucleotide of the present invention is then tested by hybridizing the filter to a radioactive probe derived from the sequence of interest by methods known in the art (Sambrook and Russell (2001) supra).

Western blot, biochemical assays and the like may be carried out on the transgenic plants to determine the presence of a polypeptide(s) encoded by the polynucleotide(s) of interest by standard procedures (Sambrook and Russell (2001) supra) using antibodies that bind to one or more epitopes present on the herbicide resistance polypeptide.

C. Methods for Selectively Controlling Weeds in a Crop Field

Methods for selectively controlling weeds in a field containing a plant are also provided. In one embodiment, the plant seeds or plants are glyphosate-resistant as a result of a polynucleotide other than SEQ ID NO:1, 13, and 38 (or any other known or published polynucleotide sequence encoding a polypeptide comprising one or more of the domains of the invention, for example SEQ ID NO:46-52) encoding a polypeptide having a Q-loop domain with increased polarity or a polynucleotide encoding a polypeptide comprising an EPSP synthase domain of the present invention being inserted into the plant seed or plant. In specific methods, the plant is treated with an effective concentration of an herbicide, where the herbicide application results in a selective control of weeds or other untransformed plants. By “effective concentration” is intended the concentration which controls the growth or spread of weeds or other untransformed plants without significantly affecting the glyphosate-resistant plant or plant seed. Such effective concentrations for herbicides of interest are generally known in the art. The herbicide may be applied either pre- or post emergence in accordance with usual techniques for herbicide application to fields comprising plants or plant seeds which have been rendered resistant to the herbicide.

D. Predicting Protein Function from Sequence

Using the methods of the invention and the identified domains, additional polypeptides (for example, SEQ ID NO:8 and 10) which confer glyphosate tolerance can be identified. These additional polypeptides can be identified by searching sequence databases containing EPSP synthase sequences, and/or by alignment of polypeptide sequences to search for the presence of domains of the present invention using methods described elsewhere herein. These polypeptides include known polypeptides as well as newly identified polypeptides. It is understood that some modification of these domains are tolerated in nature without disrupting the glyphosate resistance conferring nature of these domains, and are therefore equivalent to the domains listed herein.

In general, there are four levels of protein structure: the primary structure, which consists of the linear chain of amino acids, or the polypeptide sequence; the secondary structure, which is given by the α-helices, β-strands, and turns that the protein folds into; the tertiary structure, which is made up of simple motifs that have combined to form compact globular domains; and the quaternary structure, which can comprise several amino acid chains or subunits. When predicting function from sequence, it is important to identify the functionally important motifs or patterns. Protein domains with similar folds often share the same molecular function (Hegyi and Gerstein (1999) J. Mol. Biol. 288:147-164; Moult and Melamud (2000) Curr. Opin. Struct. Biol. 10:384-389; Shakhnovich et al. (2003) J. Mol. Biol. 326:1-9). Identification of domains important to protein function can be done by multiple sequence alignment using, for example, alignment programs described elsewhere herein.

Three-dimensional structure can be predicted by homology modeling, i.e., by using a sequence homolog (>25% sequence identity) with an experimentally determined 3D structure. The three-dimensional structure of, for example, E. coli EPSP synthase (AroA) is well known (Shönbrunn et al. (2001) Proc. Natl. Acad. Sci. USA 98:1375-1380). This structure is based on the crystallization of AroA with glyphosate and shikimate 3-phosphate.

The following examples are offered by way of illustration and not by way of limitation.

### EXPERIMENTAL

### Example 1

### Identification of Glyphosate Resistant EPSP Synthases

GRG1 is an EPSP synthase that confers glyphosate resistance upon both bacteria and plants. Comparison of the GRG1 amino acid sequence (SEQ ID NO:2) with the amino acid sequences of other glyphosate resistance EPSP synthase enzymes suggests that GRG1 is significantly different from these enzymes in the region corresponding to amino acids 90-105 of SEQ ID NO:2. This region is known to be involved in recognition of the substrate PEP (Schönbrunn et al. (2001) Proc. Natl. Acad. Sci. USA 90:1376-1380, Stauffer et al. (2001) Biochemistry 40:3951-3957). Notably, GRG1 has a motif of DCxES and a motif of PI in this region that are different from the other known glyphosate-resistant EPSP synthase enzymes. The DNA coding sequence (SEQ ID NO:1) and amino acid sequence of the grg1 open reading frame (SEQ ID NO:2) are provided in U.S. patent application Ser. No. 10/739,610, filed Dec. 18, 2003.

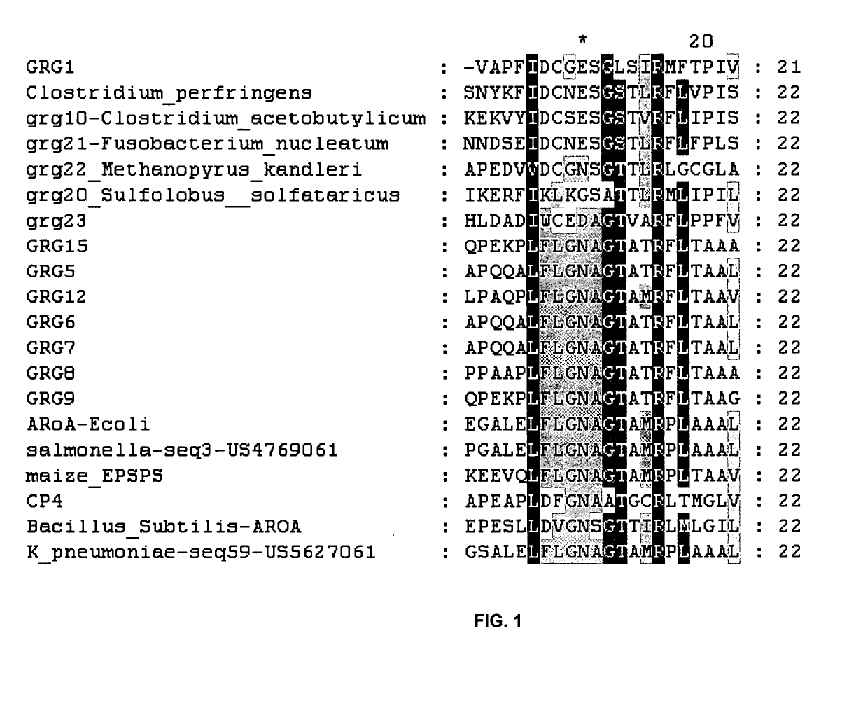

Alignment of GRG1 with other EPSP synthase enzymes and analysis of the alignment of amino acids in this Q-loop region identifies a small subset of EPSP synthase enzymes that share significant homology to GRG1 in this region of interest. Notably, the EPSP synthase enzymes from Clostridium perfringens, Clostridium acetobutylicum, Fusobacterium nucleatum, and Methanopyrus kandleri (SEQ ID NO: 4, 6, 8, and 10, respectively) are homologous to GRG1 in this region. An alignment of these proteins is provided in FIG. 1.

To test the usefulness of this novel domain to predict glyphosate resistance, and to identify novel glyphosate resistant EPSP synthase enzymes, a comparison of the amino acid sequences in this region of GRG1 was performed with a large set of published EPSP synthase amino acid sequences and several other published EPSP synthase enzymes were identified that have amino acid composition in this region similar to GRG1.

### Example 2

### Glyphosate Resistance of EPSP Synthase with Homology to GRG1 in the “Q-Loop Region”

The coding sequence of the Clostridium acetobutylicum EPSP synthase gene (SEQ ID NO:5), identified in Genbank accession number NC_{—}003030, was PCR amplified using the following primers: CAGGGATCCGCCATGAATTGTGTTAAAATAAATCCATG (upper) (SEQ ID NO:42) and CAGGGCGCGCCTTATTCCCCCAAACTCCACTC (lower) (SEQ ID NO:43). The upper primer changed the start codon to ATG from TTG, as it naturally occurs. The resultant 1.3 kb product was digested with BamH I and Asc I, and ligated into the same sites of a modified version of pUC 18 and transformed into the E. coli strain DH5a. A positive clone containing the EPSP synthase insert was identified by restriction digest and named pAX714. A pAX714 colony was struck onto minimal M63 media containing IPTG, carbenicillin and 0, 20, 50 or 100 mM glyphosate, and the plates were incubated at 37° C. The pAX714-containing cells grew very well on all concentrations of glyphosate tested, indicating that the encoded EPSP synthase was glyphosate resistant to at least 100 mM. The encoded EPSP synthase (SEQ ID NO:6) was named grg10.

### Example 3

### Cloning the EPSP Synthase Gene from Sulfolobus solfataricus

The EPSP synthase coding sequence was PCR-amplified from genomic DNA of Sulfolobus solfataricus (ATCC 35092D and SEQ ID NO:11) using the following primers:

(SEQ ID NO:44)

CAGGGATCCGCCATGATTGTAAAGATTTATCCATC (upper)
and

(SEQ ID NO:45)

CAGGGCGCGCCGGTCTCATTCAATAGAAATCTTCGC (lower).

The upper primer changed the start codon to ATG from TTG to facilitate translation in E. coli. The resultant 1.3 kb PCR product was digested with BamH I and Asc I, ligated into modified pUC18 (pAX700 backbone) which had been digested with BamH I and Asc I, then transformed into DH5α cells. A positive clone containing the EPSP synthase insert was identified by restriction digest and DNA sequencing, and named pAX716. The encoded EPSP synthase was named grg20 (SEQ ID NO:12).

### Example 4

### Testing Grg10 and Grg20 for Resistance to Glyphosate

Plasmids pAX714 and pAX716, containing grg10 and grg20, respectively, were transformed into E. coli cells and streaked onto M63 agar medium containing IPTG, carbenicillin, and various concentrations of glyphosate. Colonies of pAX701 (containing the wild-type E. coli aroA gene) were used as glyphosate-sensitive controls. The results are presented in the table below and demonstrate that expression of grg10 or grg20 confers resistance to high levels of glyphosate.

Growth of E. coli expressing grg10 or
grg20 in the presence of glyphosate.

| | | Glyphosate Concentration | | | |
|---|---|---|---|---|---|
| Plasmid | Gene | 0 mM | 20 mM | 50 mM | 100 mM |
| pAX701 | E. coli aroA | ++ | − | − | − |
| pAX714 | grg10 | ++ | +++ | +++ | +++ |
| pAX716 | grg20 | ++ | +++ | +++ | +++ |

### Example 5

### Molecular Modeling of Glyphosate-Resistant EPSP Synthases

To further identify the key domains that are predictive of glyphosate resistance, molecular modeling data was analyzed based on the published crystal structure of the E. coli EPSP synthase. First, the amino acid sequence of GRG1 was fitted to the three dimensional structure of the E. coli EPSP synthase (AroA) based on its crystallization with glyphosate and shikimate 3-phosphate (Shonbrunn et al. (2001) PNAS 98:1375-1380; Protein databank code (pdb)1G6T). The results of alteration of each of the domains of the present invention for an effect on glyphosate binding, or alteration of the substrate binding pocket was analyzed. This analysis revealed a region of interest in the loop that forms a portion of the binding pocket for PEP and its inhibitor glyphosate, and contains an invariant arginine that is known to hydrogen bond directly with phosphate of PEP. This region comprises an amino acid sequence with an increase in polarity and at least one sequence domain selected from the group consisting of:
D-C-X₁-X₂-S-G (SEQ ID NO:29), where X₁ denotes glycine, serine, alanine or asparagine, and X₂ denotes asparagine or glutamic acid; or,
D-A-X₁-X₂-S-G (SEQ ID NO:30), where X₁ denotes alanine or arginine, and X₂ denotes asparagine or glutamic acid; or,
K-L-K-X₁-S-A (SEQ ID NO:31), where X₁ denotes glycine, asparagine or glutamic acid; or,
W-C-E-D-A-G (SEQ ID NO:32).

In some embodiments, the domain residues aspartic acid, cysteine, glutamic acid and serine each have the effect of increasing the polarity of this Q-loop region. While not bound by any mechanism of action, the change in polarity in the region of EPSP synthases comprising these domains relative to other classes of EPSP synthase enzymes may result in an increase in the charge repulsion between the loop and the negatively charged phosphonate residue of glyphosate. Likewise, in some examples, the residues in this region appear to increase the bulk of this loop, and may result in steric effects that cause a downward displacement of this loop further into the binding pocket, reducing the size of the active site pocket. This effect may contribute to the reduced affinity for glyphosate observed in EPSP synthase enzymes with one or more domain(s) of the present invention. For example, GRG20 (SEQ ID NO:12) contains a substitution of two lysine residues in this loop. This substitution results in a net increase in polarity, and also results in an increased bulk due to the long side chains of the lysine residues.

Other regions of interest were identified using molecular modeling data. These regions include:
PX (SEQ ID NO:35) where X is isoleucine or leucine.

This region is present in many of the EPSP synthases with one or more domains of the present invention. The insertion of a proline at the top of the alpha helix of the Q-loop region partially unwinds the alpha helix. This insertion is likely to result in a downward displacement or other movement of the loop relative to the binding pocket, thereby affecting binding of glyphosate relative to PEP.
D-A-X₁-X₂-C-P-D-X₃-X₄-P (SEQ ID NO: 36) where X₁ is serine or threonine, X₂ is glutamine or aspartic acid, X₃ is alanine, leucine, methionine, isoleucine or valine, and X₄ is phenylalanine, alanine, leucine, methionine, isoleucine or valine, and where D is a highly conserved residue in all EPSP synthase enzymes.

Both GRG1 and GRG 10 have a conserved block of amino acids near a key aspartic acid residue of EPSP synthase. Substitution of these residues onto the E. coli structure suggests that these residues may affect the distance interaction of this key aspartic acid residue with the carbonyl end of glyphosate.

Comparison of this domain to the amino acid sequence of approximately 169 EPSP synthase enzymes suggests that, while the proline residue corresponding to position 6 of SEQ ID NO:36 is often found in EPSP synthase sequences, the cysteine residue corresponding to position 5 of SEQ ID NO:36 in combination with the proline is unique to GRG 1, GRG 10, and Clostridium perfringens EPSP synthases. Thus, the presence of this domain also appears to be associated with glyphosate resistance. LK (SEQ ID NO:37)

Several glyphosate-resistant EPSP synthase enzymes that contain a Q-loop region with an increased polarity of a domain of the present invention (including, for example, GRG1, GRG10 and EPSP synthases from Clostridium perfringens and Fusobacterium nucleatum) also contain a conserved LK domain. Analysis of the location of this sequence by fitting on the E. coli crystal structure shows that this sequence is exposed to the exterior surface of the molecule. Since this sequence is not close to any known key regions of EPSP synthases, and does not seem to be directly involved in binding of PEP, glyphosate, or shikimate 3-phosphate, the contribution of this sequence to glyphosate resistance is not yet known. Further, since this domain is found in many EPSP synthase enzymes other than those containing domains of the present invention, this sequence may have little or no effect on glyphosate resistance in the absence of a Q-loop region having an increased polarity or of a presently described domain. It may however, affect other properties of the protein.

### Example 6

### Prediction of Additional Glyphosate-Resistant Enzymes Comprising Domains of the Present Invention

Given the discovery of these key domains, we were able to predict the existence of several glyphosate resistant EPSP synthase enzymes.

The EPSP synthase from Fusobacterium nucleatum and Methanopyrus kandleri are highly homologous to both GRG1 and GRG10 in the Q-loop region, and thus were predicted to confer glyphosate resistance on cells.

### Example 7

### Cloning the EPSP Synthase Gene from Fusobacterium nucleatum Subsp nucleatum

The published amino acid sequence of the Fusobacterium nucleatum EPSP synthase (SEQ ID NO:7) was obtained from GENBANKS and designed synthetically by backtranslation and synthesized in vitro using DNA 2.0. The resultant DNA sequences were designed to include flanking BamH I and Asc I sites to facilitate subcloning. The synthetic gene was excised from DNA2.0's donor vector using BamH I and Asc I, gel purified, ligated into the same sites of a modified pUC 18 which had been digested with BamH I and Asc I, then transformed into DH5α cells. A positive clone containing the EPSP synthase insert was identified by restriction digest and DNA sequencing, and named pAX723 (synFusoII). The encoded EPSP synthase was named grg21 (SEQ ID NO:8).

### Example 8

### Cloning the EPSP Synthase Gene from Methanopyrus kandleri

The published amino acid sequence of the Methanopyrus kandleri EPSP synthase was obtained from GENBANK® and designed synthetically by backtranslation and synthesized in vitro using DNA 2.0. The resultant DNA sequence (SEQ ID NO:9) was designed to include flanking BamH I and Asc I sites to facilitate subcloning. The synthetic gene was excised from DNA2.0's donor vector using BamH I and Asc I, gel purified, ligated into the same sites of a modified pUC18 which had been digested with BamH I and Asc I, then transformed into DH5α cells. A positive clone containing the EPSP synthase insert was identified by restriction digest and DNA sequencing, and named pAX724 (synMethII). The encoded EPSP synthase was named grg22 (SEQ ID NO:10).

### Example 9

### Testing Grg21 and Grg22 for Resistance to Glyphosate

Plasmids pAX723 and pAX72⁴, containing grg21 and grg22, respectively, were transformed into E. coli cells and streaked onto M63 agar medium containing IPTG, carbenicillin, and various concentrations of glyphosate. Colonies of pAX701 (containing the wild-type E. coli aroA gene) were used as glyphosate-sensitive controls. The results are presented in the table below. Expression of grg21 or grg22 confers resistance to high levels of glyphosate.

Growth of E. coli expressing grg21 or
grg22 in the presence of glyphosate.

| | | Glyphosate Concentration | | | |
|---|---|---|---|---|---|
| Plasmid | Gene | 0 mM | 20 mM | 50 mM | 100 mM |
| pAX701 | E. coli aroA | ++ | − | − | − |
| pAX723 | grg21 | ++ | +++ | +++ | +++ |
| pAX724 | grg22 | ++ | +++ | +++ | +++ |

### Example 10

### GRG23 Contains a Glyphosate-Resistant EPSP Synthase Domain

GRG23 (U.S. Patent Application No. 60/741,166, filed Dec. 1, 2005 and SEQ ID NO:14) was isolated from a bacterial strain exhibiting strong glyphosate resistance. GRG23 comprises an EPSP synthase domain of the present invention that has an increased polarity in the Q-loop region relative to EPSP synthase enzymes not containing a domain of the present invention. This enzyme confers glyphosate tolerance to an organism transformed with an expression construct expressing GRG23.

### Example 11

### Potential for Proteins with Combinations of Domains

The domains provided herein do not overlap with respect to the previously defined Class II (U.S. Pat. No. 5,627,061) or Class III (U.S. Patent Application No. 60/695,193, filed Jun. 29, 2005) EPSP synthase domains. Thus, it is conceivable that a protein may exist in nature that would contain all or some elements of both the domains of the present invention and Class II or Class III domains (for example, the EPSP synthase derived from Clostridium tetani (Swissprot accession number Q894D2 and SEQ ID NO:28) contains both Class II and domains of the present invention).

In some embodiments of the present invention, the presence of a domain of the present invention in an EPSP synthase enzyme is predictive of glyphosate resistance. In further embodiments, the presence of all or part of that domain is associated with an increase or enhancement in enzyme activity or function. In another embodiment, the domains identified herein may be engineered or recombined with the amino acid sequences of other enzymes, for example, by replacement of a Class I EPSP synthase motif of the E. coli aroA gene with a polypeptide having a Q-loop region with increased polarity, or with all or part of a domain of the present invention. Alternatively, one or more domain(s) of the present invention may be inserted in replace of a polypeptide that does not comprise a domain of the present invention (including Class I and Class II EPSP synthase polypeptides), which may or may not comprise or result in improved properties.

### Example 12

### Identification of Additional Novel EPSP Synthase Enzymes

Using the methods of the invention, one can identify further glyphosate resistant EPSP synthases by searching databases containing EPSP synthase enzymes, and/or by alignment of the amino acid sequence of EPSP synthase enzymes and analysis for proteins containing a Q-loop region with increased polarity or domains of the present invention. It is understood that some modification of this Q-loop region or these domains is tolerated in nature without disrupting the glyphosate resistance conferring nature of these regions, and are therefore equivalent to the domains listed herein. Therefore, it is recognized that enzymes having about 90%, about 91%, about 92%, about 93%, about 94%, about 95%, about 96%, about 97%, about 98%, about 99% or greater homology to a domain of the invention could confer glyphosate tolerance.

Given the invention, it is now possible to generate further EPSP synthase enzymes with alterations in the Q-loop region that confer glyphosate resistance, in some instances without generating primary amino acid similarity to the specific domain residues described herein. For example, one may in general increase the polarity in the Q-loop region, and/or increase the bulkiness of the residues in this region, and achieve a similar glyphosate resistant EPSP synthase. Some of these alterations generated by use of the invention are likely to improve the glyphosate tolerance of the resulting protein, and are incorporated herein. Thus, the invention encompasses the modification of EPSP synthase amino acid sequences to increase polarity, bulkiness, or to contain a domain of the invention.

In another embodiment of the invention, the domains identified herein may be engineered or recombined with the amino acid sequences of other EPSP synthase enzymes. For example, one of more of the domain sequences described herein may be inserted into an EPSP synthase sequence not containing a domain of the present invention. The resulting proteins may have altered as well as improved properties.

### Example 13

### Plant Transformation by Particle Bombardment

Maize ears are best collected 8-12 days after pollination. Embryos are isolated from the ears, and those embryos 0.8-1.5 mm in size are preferred for use in transformation. Embryos are plated scutellum side-up on a suitable incubation media, such as DN62A5S media (3.98 g/L N6 Salts; 1 ml/L (of 1000× Stock) N6 Vitamins; 800 mg/L L-Asparagine; 100 mg/L Myo-inositol; 1.4 g/L L-Proline; 100 mg/L Casamino acids; 50 g/L sucrose; 1 ml/L (of I mg/ml stock) 2,4-D). However, media and salts other than DN62A5S are suitable and are known in the art. Embryos are incubated overnight at 25° C. in the dark. However, it is not necessary per se to incubate the embryos overnight.

The resulting explants are transferred to mesh squares (30-40 per plate), transferred onto osmotic media for about 30-45 minutes, then transferred to a beaming plate (see, for example, PCT Publication No. WO/0138514 and U.S. Pat. No. 5,240,842).

DNA constructs designed to express EPSP synthase sequences having a Q-loop with an increased polarity or containing a domain of the present invention in plant cells are accelerated into plant tissue using an aerosol beam accelerator, using conditions essentially as described in PCT Publication No. WO/0138514. After beaming, embryos are incubated for about 30 min on osmotic media, and placed onto incubation media overnight at 25° C. in the dark. To avoid unduly damaging beamed explants, they are incubated for at least 24 hours prior to transfer to recovery media. Embryos are then spread onto recovery period media, for about 5 days, 25° C. in the dark, then transferred to a selection media. Explants are incubated in selection media for up to eight weeks, depending on the nature and characteristics of the particular selection utilized. After the selection period, the resulting callus is transferred to embryo maturation media until the formation of mature somatic embryos is observed. The resulting mature somatic embryos are then placed under low light, and the process of regeneration is initiated by methods known in the art. The resulting shoots are allowed to root on rooting media, and the resulting plants are transferred to nursery pots and propagated as transgenic plants. The plants are assayed for improved resistance to glyphosate.

Materials
DN62A5S Media

| Components | per liter | | Source |
|---|---|---|---|
| Chu's N6 Basal Salt | 3.98 | g/L | Phytotechnology Labs |
| Mixture (Prod. No. C 416) | | | |
| Chu's N6 Vitamin Solution | 1 | ml/L | Phytotechnology Labs |
| (Prod. No. C 149) | (of 1000× Stock) | | |
| L-Asparagine | 800 | mg/L | Phytotechnology Labs |
| Myo-inositol | 100 | mg/L | Sigma |
| L-Proline | 1.4 | g/L | Phytotechnology Labs |
| Casamino acids | 100 | mg/L | Fisher Scientific |
| Sucrose | 50 | g/L | Phytotechnology Labs |
| 2,4-D (Prod. No. D-7299) | 1 | ml/L | Sigma |

(of 1 mg/ml Stock)

Adjust the pH of the solution to pH 5.8 with 1N KOH/1N KCl, add Gelrite (Sigma) to 3 g/L, and autoclave. After cooling to 50° C., add 2 ml/L of a 5 mg/ml stock solution of Silver Nitrate (Phytotechnology Labs). Recipe yields about 20 plates.

### Example 14

### Transformation of Plant Cells by Aarobacterium-Mediated Transformation

Ears are best collected 8-12 days after pollination. Embryos are isolated from the ears, and those embryos 0.8-1.5 mm in size are preferred for use in transformation. Embryos are plated scutellum side-up on a suitable incubation media, and incubated overnight at 25° C. in the dark. However, it is not necessary per se to incubate the embryos overnight. Embryos are contacted with an Agrobacterium strain containing the appropriate vectors having a\n EPSP synthase enzyme with a Q-loop region with an increased polarity or a domain of the present invention for Ti plasmid mediated transfer for about 5-10 min, and then plated onto co-cultivation media for about 3 days (25° C. in the dark). After co-cultivation, explants are transferred to recovery period media for about five days (at 25° C. in the dark). Explants are incubated in selection media for up to eight weeks, depending on the nature and characteristics of the particular selection utilized. After the selection period, the resulting callus is transferred to embryo maturation media, until the formation of mature somatic embryos is observed. The resulting mature somatic embryos are then placed under low light, and the process of regeneration is initiated as known in the art. The resulting shoots are allowed to root on rooting media, and the resulting plants are transferred to nursery pots and propagated as transgenic plants.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

That which is claimed:

1. An isolated polynucleotide other than the polynucleotide of SEQ ID NO:1, 3, 5, 11, 13, 38 or 40 encoding an EPSP synthase polypeptide having a Q-loop, said Q-loop comprising an amino acid sequence with an increased polarity, wherein said polypeptide is resistant to glyphosate.

2. The polynucleotide of claim 1, wherein said Q-loop has at least one sequence domain selected from the group consisting of:
a) D-C-X₁-X₂-S-G (SEQ ID NO:29), where X₁ denotes glycine, serine, alanine or asparagine, and X₂ denotes asparagine or glutamic acid;
b) D-A-X₁-X₂-S-G (SEQ ID NO:30), where X₁ denotes alanine or arginine, and X₂ denotes asparagine or glutamic acid;
c) K-L-K-X₁-S-A (SEQ ID NO:31), where X₁ denotes glycine, asparagine or glutamic acid; or,
d) W-C-E-D-A-G (SEQ ID NO:32).

3. The polynucleotide of claim 1, wherein said Q-loop has at least a serine or threonine corresponding to amino acid residue 98 of SEQ ID NO:22.

4. A polynucleotide of claim 1 in which the polynucleotide encodes a fusion polypeptide comprising an amino-terminal chloroplast transit peptide and the EPSP synthase enzyme.

5. A method of producing genetically transformed plants which are tolerant toward glyphosate herbicide, comprising the steps of:
a) inserting into the genome of a plant cell a polynucleotide other than the polynucleotide of SEQ ID NO:1, 13 or 38 encoding a polypeptide having a Q-loop, said Q-loop comprising an amino acid sequence with an increased polarity;
b) obtaining a transformed plant cell; and,
c) regenerating from the transformed plant cell a genetically transformed plant which has increased tolerance to glyphosate herbicide.

6. The method of claim 5, wherein said Q-loop has at least one sequence domain selected from the group consisting of:
a) D-C-X₁-X₂-S-G (SEQ ID NO:29), where X₁ denotes glycine, serine, alanine or asparagine and X₂ denotes asparagine or glutamic acid;
b) D-A-X₁-X₂-S-G (SEQ ID NO:30), where X₁ denotes alanine or arginine, and X₂ denotes asparagine or glutamic acid;
c) K-L-K-X₁-S-A (SEQ ID NO:31), where X₁ denotes glycine, asparagine or glutamic acid; or,
d) W-C-E-D-A-G (SEQ ID NO:32).

7. The method of claim 5, wherein said Q-loop has at least a serine or threonine corresponding to amino acid residue 98 of SEQ ID NO:22.

8. A method of claim 5 in which the polynucleotide encodes a fusion polypeptide comprising an amino-terminal chloroplast transit peptide and the EPSP synthase enzyme.

9. A glyphosate tolerant plant cell comprising a heterologous polynucleotide other than the polynucleotide of SEQ ID NO:1, 13 or 38 encoding an EPSP synthase polypeptide having a Q-loop, said Q-loop comprising an amino acid sequence with an increased polarity, wherein said polypeptide is resistant to glyphosate.

10. The glyphosate tolerant plant cell of claim 9, wherein said Q-loop has at least one sequence domain selected from the group consisting of:
a) D-C-X₁-X₂-S-G (SEQ ID NO:29), where X₁ denotes glycine, serine, alanine or asparagine, and X₂ denotes asparagine or glutamic acid;
b) D-A-X₁-X₂-S-G (SEQ ID NO:30), where X₁ denotes alanine or arginine, and X₂ denotes asparagine or glutamic acid;
c) K-L-K-X₁-S-A (SEQ ID NO:31), where X₁ denotes glycine, asparagine or glutamic acid; or,
d) W-C-E-D-A-G (SEQ ID NO:32).

11. The glyphosate tolerant plant cell of claim 9, wherein said Q-loop has at least a serine or threonine corresponding to amino acid residue 98 of SEQ ID NO:22.

12. The glyphosate tolerant plant cell of claim 9 in which the polynucleotide encodes a fusion polypeptide comprising an amino-terminal chloroplast transit peptide and the EPSP synthase enzyme.

13. The glyphosate tolerant plant cell of claim 9 selected from the group consisting of corn, wheat, rice, barley, soybean, cotton, sugarbeet, oilseed rape, canola, flax, sunflower, potato, tobacco, tomato, alfalfa, poplar, pine, eucalyptus, apple, lettuce, peas, lentils, grape and turf grasses.

14. A glyphosate tolerant plant comprising the plant cell of claim 9.

15. Transformed seed of the plant of claim 14.

16. The glyphosate tolerant plant of claim 14 selected from the group consisting of corn, wheat, rice, barley, soybean, cotton, sugarbeet, oilseed rape, canola, flax, sunflower, potato, tobacco, tomato, alfalfa, poplar, pine, eucalyptus, apple, lettuce, peas, lentils, grape and turf grasses.

17. A method for selectively controlling weeds in a field containing a plant having planted seeds or plants comprising the steps of:
a) planting the seeds or plants which are glyphosate tolerant as a result of a polynucleotide other than the polynucleotide of SEQ ID NO:1, 13, or 38 being inserted into the seed or plant, said polynucleotide having a Q-loop, said Q-loop comprising an amino acid sequence with an increased polarity; and,
b) applying to the plants and weeds in a field an effective concentration of glyphosate herbicide to control weeds without significantly affecting the plants.

18. The method of claim 17, wherein said Q-loop has at least one sequence domain selected from the group consisting of:
a) D-C-X₁-X₂-S-G (SEQ ID NO:29), where X₁ denotes glycine, serine, alanine or asparagine and X₂ denotes asparagine or glutamic acid;
b) D-A-X₁-X₂-S-G (SEQ ID NO:30), where X₁ denotes alanine or arginine, and X₂ denotes asparagine or glutamic acid;
c) K-L-K-X₁-S-A (SEQ ID NO:3 1), where X₁ denotes glycine, asparagine or glutamic acid; or,
d) W-C-E-D-A-G (SEQ ID NO:32).

19. The method of claim 17, wherein said Q-loop has at least a serine or threonine corresponding to amino acid residue 98 of SEQ ID NO:22.

20. The method of claim 17 in which the polynucleotide encodes a fusion polypeptide comprising an amino terminal chloroplast transit peptide and the EPSP synthase enzyme.


SEQUENCE LISTING (52 sequences)

SEQ ID NO: 1 - DNA, 1398 bp, Enterobacteriaceae sp.
  CDS (103)...(1398)
aaaaaaggaaatgaactatgtgttgctggaaaaagtagggaagggagtggtgaagagtattccactggttcaattagaaaaaatcattcaaggattaccaaagtgaaagtaacaatacagcccggagatctgactggaattatccagtcacccgcttcaaaaagttcgatgcagcgagcttgtgctgctgcactggttgcaaaaggaataagtgagatcattaatcccggtcatagcaatgatgataaagctgccagggatattgtaagccggcttggtgccaggcttgaagatcagcctgatggttctttgcagataacaagtgaaggcgtaaaacctgtcgctccttttattgactgcggtgaatctggtttaagtatccggatgtttactccgattgttgcgttgagtaaagaagaggtgacgatcaaaggatctggaagccttgttacaagaccaatggatttctttgatgaaattcttccgcatctcggtgtaaaagttaaatctaaccagggtaaattgcctctcgttatacaggggccattgaaaccagcagacgttacggttgatgggtccttaagctctcagttccttacaggtttgttgcttgcatatgcggccgcagatgcaagcgatgttgcgataaaagtaacgaatctcaaaagccgtccgtatatcgatcttacactggatgtgatgaagcggtttggtttgaagactcccgagaatcgaaactatgaagagttttatttcaaagccgggaatgtatatgatgaaacgaaaatgcaacgatacaccgtagaaggcgactggagcggtggtgcttttttactggtagcgggggctattgccgggccgatcacggtaagaggtttggatatagcttcgacgcaggctgataaagcgatcgttcaggctttgatgagtgcgaacgcaggtattgcgattgatgcaaaagagatcaaacttcatcctgctgatctcaatgcatttgaatttgatgctactgattgcccggatctttttccgccattggttgctttggcgtcttattgcaaaggagaaacaaagatcaaaggcgtaagcaggctggcgcataaagaaagtgacagaggattgacgctgcaggacgagttcgggaaaatgggtgttgaaatccaccttgagggagatctgatgcgcgtgatcggagggaaaggcgtaaaaggagctgaagttagttcaaggcacgatcatcgcattgcgatggcttgcgcggtggctgctttaaaagctgtgggtgaaacaaccatcgaacatgcagaagcggtgaataaatcctacccggatttttacagcgatcttaaacaacttggcggtgttgtatctttaaaccatcaatttaatttctcatga

SEQ ID NO: 2 - protein, 431 aa, Enterobacteriaceae sp.
VKVTIQPGDLTGIIQSPASKSSMQRACAAALVAKGISEIINPGHSNDDKAARDIVSRLGARLEDQPDGSLQITSEGVKPVAPFIDCGESGLSIRMFTPIVALSKEEVTIKGSGSLVTRPMDFFDEILPHLGVKVKSNQGKLPLVIQGPLKPADVTVDGSLSSQFLTGLLLAYAAADASDVAIKVTNLKSRPYIDLTLDVMKRFGLKTPENRNYEEFYFKAGNVYDETKMQRYTVEGDWSGGAFLLVAGAIAGPITVRGLDIASTQADKAIVQALMSANAGIAIDAKEIKLHPADLNAFEFDATDCPDLFPPLVALASYCKGETKIKGVSRLAHKESDRGLTLQDEFGKMGVEIHLEGDLMRVIGGKGVKGAEVSSRHDHRIAMACAVAALKAVGETTIEHAEAVNKSYPDFYSDLKQLGGVVSLNHQFNFS

SEQ ID NO: 3 - DNA, 1275 bp, Clostridium perfringens
gtgaaaaaggtaattataactcctagtaagttaaggggaagtgtaaaaataccaccttctaaaagtatggctcatagagctattatttgtgcttctttaagcaaaggagaaagtgttatttctaacatagatttttcagaagatattattgcaactatggaaggtatgaaatctttaggagcaaatataaaagtagaaaaagataaactaattataaatggagaaaatattttaaaggattctaattataaatttattgattgtaatgaatcaggttccactttaagatttttagttccaatttccttaataaaagataatagagttaattttatcggtagaggaaatttagggaaaagaccattaaaaacttattatgagatttttgaggagcaagaaattaagtattcctatgaggaagaaaatcttgatttgaatatagaaggaagcttaaaaggtggagaattcaaagttaagggaaatataagttctcaatttataagtggtttattatttactcttcctttattaaaagatgattctaaaataataataactacagaacttgaatctaaaggatatatagatttaactttagacatgatagaaaagtttggagttacaataaaaaataataattatagagaatttttaataaaaggtaatcaaagttataagcctatgaattataaggttgaaggtgattactcacaggctgctttctatttttcagcaggggccttaggctcagaaataaattgtcttgatttagatttaagttcttatcaaggagataaggaatgcattgaaatattagagggtatgggtgctaggcttatagaaagtcaagaaaggtctttaagtataattcatggggatttaaatggaacaattatagatgcttcacaatgcccagatataattcctgttttgacagtggttgctgctttaagtaagggagagactaggattataaatggagaaagacttagaataaaagaatgtgatagattaaatgctatatgtacagagcttaataaactaggtgcagatataaaggaattaaaagatggacttataataaatggagttaaagatttaataggaggagaagtatatagccataaagatcatagaatagctatgagtttggctattgcttctacaagatgcaagaaagaggttattataaaagaaccagattgtgttaaaaaatcttatccaggattttgggaagattttaagagcttaggtggaattttaagagaagaataa

SEQ ID NO: 4 - protein, 424 aa, Clostridium perfringens
MKKVIITPSKLRGSVKIPPSKSMAHRAIICASLSKGESVISNIDFSEDIIATMEGMKSLGANIKVEKDKLIINGENILKDSNYKFIDCNESGSTLRFLVPISLIKDNRVNFIGRGNLGKRPLKTYYEIFEEQEIKYSYEEENLDLNIEGSLKGGEFKVKGNISSQFISGLLFTLPLLKDDSKIIITTELESKGYIDLTLDMIEKFGVTIKNNNYREFLIKGNQSYKPMNYKVEGDYSQAAFYFSAGALGSEINCLDLDLSSYQGDKECIEILEGMGARLIESQERSLSIIHGDLNGTIIDASQCPDIIPVLTVVAALSKGETRIINGERLRIKECDRLNAICTELNKLGADIKELKDGLIINGVKDLIGGEVYSHKDHRIAMSLAIASTRCKKEVIIKEPDCVKKSYPGFWEDFKSLGGILREE

SEQ ID NO: 5 - DNA, 1398 bp, Clostridium acetobutylicum; identical to SEQ ID NO: 1

SEQ ID NO: 6 - protein, 428 aa, Clostridium acetobutylicum
MNCVKINPCCLKGDIKIPPSKSLGHRAIICAALSEEESTIENISYSKDIKATCIGMSKLGALIIEDAKDNSTLKIKKQKLVSKEKVYIDCSESGSTVRFLIPISLIEERNVVFDGQGKLSYRPLDSYFNIFDEKEIAYSHPEGKVLPLQIKGRLKAGMFNLPGNISSQFISGLMFSLPFLEGDSIINITTNLESVGYVDMTIDMLKKFGIEIENKAYKSFFIKGNQKCKGTKYKVEGDFSQAAFWLSAGILNGNINCKDLNISSLQGDKVILDILKKMGGAIDEKSFSSKKSHTHGIVIDASQCPDLVPILSVVAALSEGTTKIVNAARLRIKESDRLKAMATELNKLGAEVVELEDGLLIEGKEKLKGGEVESWNDHRIAMALGIAALRCEESVTINGSECVSKSYPQFWSDLKQLGGDVHEWSLGE

SEQ ID NO: 7 - DNA, 1309 bp, Artificial Sequence
  Backtranslated from a protein isolated from Fusobacterium nucleatum
  CDS (10)...(1284)
  misc_feature (0)...(0): synFuso II
ggatccggcatgaggaacatgaacaagaagatcatcaaggcggataagctcgtcggcgaggtcacccccccccccagcaagtcagtcctgcatcgttacatcatcgcctccagcctggcgaagggtatctccaagatcgagaacatcagctactccgatgatatcatcgccaccatcgaggcgatgaagaagctgggcgccaacatcgagaagaaggataactacctcctgatcgatggcagcaagaccttcgataaggagtacctcaacaacgattcagagatcgattgcaacgagtccggcagcaccctgcgcttcctcttccccctgagcatcgtcaaggagaacaagatcctgttcaagggcaagggcaagctgttcaagcgccccctctccccctacttcgagaacttcgataagtaccagatcaagtgcagcagcatcaacgagaacaagatcctcctggatggcgagctcaagtcaggcgtctacgagatcgatggcaacatcagcagccagttcatcaccggcctgctcttcagcctccccctgctcaacggcaactccaagatcatcatcaagggcaagctcgagagcagctcctacatcgatatcacccttgattgcctgaacaagttcggcatcaacatcatcaacaactcatacaaggagttcatcatcgagggcaaccagacctacaagtccggcaactaccaggtcgaggcggattacagccaggtcgccttcttcctggtcgccaactccatcggctccaacatcaagatcaacggcctcaacgtcaactccctccagggcgataagaagatcatcgatttcatctcagagatcgataactggaccaagaacgagaagctgatcctcgatggcagcgagacccccgatatcatccccatcctgagcctcaaggcgtgcatcagcaagaaggagatcgagatcgtcaacatcgcccgcctccgcatcaaggagtccgatcgcctgtcagcgaccgttcaagagctctccaagctcggcttcgatctgatcgagaaggaggattccatcctgatcaactcccgcaagaacttcaacgagatcagcaacaactcccccatcagcctcagctcacatagcgatcatcgtatcgccatgaccgtcgccatcgcgtccacctgctacgagggcgagatcatcctggataacctcgattgcgtcaagaagagctaccctaacttctgggaggttttcctcagcctgggcggcaagatctacgagtacctcggctgaggcgcgcctgcaggtcgacaagctt

SEQ ID NO: 8 - protein, 424 aa, Fusobacterium nucleatum
MRNMNKKIIKADKLVGEVTPPPSKSVLHRYIIASSLAKGISKIENISYSDDIIATIEAMKKLGANIEKKDNYLLIDGSKTFDKEYLNNDSEIDCNESGSTLRFLFPLSIVKENKILFKGKGKLFKRPLSPYFENFDKYQIKCSSINENKILLDGELKSGVYEIDGNISSQFITGLLFSLPLLNGNSKIIIKGKLESSSYIDITLDCLNKFGINIINNSYKEFIIEGNQTYKSGNYQVEADYSQVAFFLVANSIGSNIKINGLNVNSLQGDKKIIDFISEIDNWTKNEKLILDGSETPDIIPILSLKACISKKEIEIVNIARLRIKESDRLSATVQELSKLGFDLIEKEDSILINSRKNFNEISNNSPISLSSHSDHRIAMTVAIASTCYEGEIILDNLDCVKKSYPNFWEVFLSLGGKIYEYLG

SEQ ID NO: 9 - DNA, 1321 bp, Artificial Sequence
  Backtranslated from a protein isolated from Methanopyrus kandleri
  CDS (10)...(1296)
  misc_feature (0)...(0): synMeth II
ggatccggcatgaagcgcgtcgagttggagggtatccctgaggtccgtggtactgtctgccctcctccttctaagagcggttctcatcgcgccttgatcgccgcgtctttgtgcgatggttcaaccgagttgtggaacgtcctggatgcggaggatgtccgtgcgactttgcgtctgtgccgtatgttgggtgccgaggttgatgtcgatggtgaggagcgtttggaggcgactgtttccggtttcggtgattccccccgtgcgcctgaggatgttgttgattgcggcaacagcggtaccaccttgaggctcggttgcggtttggcggccttggttgagggtactactatcctcaccggtgatgatagcctccgttccaggcctgttggtgatctgctggccgccttgcgttcattgggtgttgatgcccgtggtcgtgttgttcgtggtgaggagtaccctcctgttgtcatcagcggtaggcctctgagggagagggttgcggtttacggtgatgtctcctctcagttcgtcagcgccttgctgttcctgggtgcgggtttgggtgccttgagggttgatgttgtcggtgatctgcgttcccgtccttacgttgatatgaccgtcgagaccctcgagaggttcggtgtcagcgtcgttagggagggttcctctttcgaggtcgagggtcgtcctcgttcacctggtaagctgagggtcgagaacgattggtcctccgccggttacttcgttgcgttgggtgcgatcggtggtgagatgcgtatcgagggtgttgatctggatagcagccatcccgatcgtaggatcgtcgagatcacccgcgagatgggtgccgaggttcgtcgtatcgatggtggtatcgtcgtccgttcaaccggtcgtttggagggtgttgaggtcgatctgagcgattcccctgatctggtccctaccgtcgcggccatggcctgcttcgccgagggtgttactcgtatcgagaacgttggtcatttgaggtacaaggaggtcgatcgcctgcgtgcgttggccgcggagttgcctaagttcggtgttgaggttagggagggtaaggattggttggagatagtcggtggtgagcctgttggtgccagggttgattcaaggggtgatcataggatggcgatggcgctggcggttgttggtgcgttcgccaggggtaagaccgttgttgagcgtgccgatgcggtttcaatctcttaccccaggttctgggaggatctcgcctctgttggtgtccctgttcattccgtttgaggcgcgcctgcaggtcgacaagctt

SEQ ID NO: 10 - protein, 428 aa, Methanopyrus kanleri
MKRVELEGIPEVRGTVCPPPSKSGSHRALIAASLCDGSTELWNVLDAEDVRATLRLCRMLGAEVDVDGEERLEATVSGFGDSPRAPEDVVDCGNSGTTLRLGCGLAALVEGTTILTGDDSLRSRPVGDLLAALRSLGVDARGRVVRGEEYPPVVISGRPLRERVAVYGDVSSQFVSALLFLGAGLGALRVDVVGDLRSRPYVDMTVETLERFGVSVVREGSSFEVEGRPRSPGKLRVENDWSSAGYFVALGAIGGEMRIEGVDLDSSHPDRRIVEITREMGAEVRRIDGGIVVRSTGRLEGVEVDLSDSPDLVPTVAAMACFAEGVTRIENVGHLRYKEVDRLRALAAELPKFGVEVREGKDWLEIVGGEPVGARVDSRGDHRMAMALAVVGAFARGKTVVERADAVSISYPRFWEDLASVGVPVHSV

SEQ ID NO: 11 - DNA, 13066 bp, Sulfolobus solfataricus
  misc_feature (0)...(0): aroA coding sequence 10,578 - 11,822
ctgcagtatttctaactgtcattaaatcgtttggttttgtaactctcatctttattacaatctcaaactctttgccttctactttgctttcaattgacattccttctggcaaatttacatcatcaacttttaaacttcttataaatagttctgcatccttatattccgatgagattcttaactctatcatcaattttaatcaatccttctttctttactgacggaataaaaaatctactagtgaaaagtctatcattgtctctgattactatcattttatctctccttattttttgtaactgtaacaatattatttgtgctaagagaggagaagtgagattagatgtctcaacaatataatatgactgtttattttcaatagaattgattgagaatcccttggagagagtttgcctaaatctactaattaatgcctctgcataagaggatgacaggacaaactgtaagatctcattactgccaatagtatcaaacgaataaattaaagagaatgccaactctaatatatcataatcaagataaaaaaatctatccgtaattatatcatccctagtgaattttggattttctttcacaatagtagaaattattctgaaaagtaatgtatttagctgactctcattcaaatcttctaatttcgatatctcactagcgtttacctctcttatgagacttatggcattttctctattgccagatatgccaggtatataaggatctagagaaaacattaacgataaaaaggctggtaaagttttatatgcaggaattttcagatttttttcgatagtcatatcaaatttctccatttcccttattgtctctaattcccattcggaaagtgatcttctgtctaatattgtagacgttataatacaactaacaattggcaagacatcttcagcagttataggtaaatatgaacaaaaggaattctgacccaagaaatattttttcttattatctattatccatttcccgttagcgtcaagtctaatttgtgtttcacattcttctggaatgaacgaaagagtgaaagaattcttagtatttttggaaataataaatgaaaataggataggctctatagaatagtacgcagataaacacaaattatccttagaatacacattcctaactacttcccttatttcttgattattcggctctcctaagaatacttccattactatctctttagaatttcttttgaccattttttaagctcttcaaagctcatatccttaaagaaaatagatccaagcaagtttgatataatatagttcaatcttgtagcaaagacgtaagagggatttaaaaatattgataacgctaattctcccactattgctgcagatggtatcggtattaatatagatacttggtataaaaaagagataaaaattgacaagaaaatattatgtgttacataataaaaataacctccatataaggcaaaactaaatatatcaatcatgatataaattacaaaatcctttgtggttccttgcttaaggtaatatttaaattccatataatctgtaacaattcgttccattttaactatcttaaagataaattgttcaattttatttactttatcatgttgcaagtaaaaatacgacaatgctcctatccatccagctatgttaaataatacgactagaatgaatacaaattctaatgggttaaaaaagaatggcagcagtaaaatatagcccaaacatattgcgagcacatctatggatcctataagaatagagtagctaaacgcttgccttaagtttaccccatatttgttgtaaactaaacttctaaccaattcttgtccagcccatccaggtactaataaacctacgaagttacctagtaatcttgctcttaacgttatatctactctacgcttaattagtaatgaatccttaaatgatgagacgaaattctgggcagtatacgttaaaagaaaaatcagaaagaatttgggatcttcttgtaaaacatatattatattaatcttaaatatgtatgcatagactatgatcactataaacggaagaaaaatagctgcaatgtatttcttatccattatcttccccttttctcaaaacatccaatactaggatagcataaagttacattcccaagatttttaaacactttgggctcatctaactcaggaattttcatattagttctaaatattccacatttaggtttacttatcttttgatatagaaaaggatactcatttagaacgcatgtcttacattgtgagttcttctcgatgttaattctctcaatttttaattctctagaatctatatagaataatgaataatccggattgcctctcaagtgattaagcattaagttaacttgaagtgtagctgttaattctactattagtggagtagttccaataacatcacatgaattcccaatttcgtcttgatctgaatagtcaataaaacaagataaacaggaggtttgactagggtctatcagtttagcagaaccgtattcaccattaattcctccatatattaggatttttcctaattttactatagcatcatttaacaatagcttgtagtacaagctgtctaatgcatcgaacacgtaatccttatcagaaattagcctctcgacgttctcctcgtcgagtatatctataatataattaattttgattgaagaatttataagggatatttttttagcgcaaacttcagctttaggtttgcctacatcattttcatcaaataaatggaccctatgtagattagttatatctaccacatctgcatctactatagttaactctttaactcctagcctagctagcaactctgcaacggcagtacctaaagctccacagcctgcaattaatatctttaactcatttaacctctgttgaattcctaatcctaaaactattagttgcctagaatatctttccacaagattataatgtagaataatctttaaaaataagtgttgcctactaaaagtggggattgatattgttccaatggaaaaaggtggagggagtgatggaactccaatatcaatagaggaattggacaagttaagacaagtagctgaaaaggcaagaagaaatgtaataaaaatgctattttatgatcaaacaatacatgtgggatcgtccctaagtagcatagagatattaactacgttaatattcaagcatataaggacggattcaagcttagtgaataaagactggcttattttaagtaaaggccatgcagcgccagctctttatgccgttttagctgaaaaaggttacataaaagaagaggaactttggagaatacaagatataacgggattattacaagggcatccagaaacttttattcccggtgtagatatgtcgactggtagtttagggcaaggtttgagctttggaataggtgttgctactggtataaagatggccaacggcactggaagagtatatgtcataatgggtgatggtgaacaggatgagggagaaatatgggaggctatgacgcatgcagtagttagaaatcttgataacttaattgcatttatagagatgaacaatttccagcttgatggttcaacagatgagataaaaccaaagaacttcttacctaaggtatgggaagcagtaggttggaaagtattaaactgcgatgggcatgatttcattagtattactaatgcagttaacgaggcatataaggcaagcaagcccgtagtaatattcgctaagactgtaagaggaaaggggtttcctccaatagaaaatacccataaacagaggtccagtccagatgatgcaaggaaatatttactcaatgcgtgaaaccttcggaaggctattagcagacctaggggataagaacaaggatctagtcgtgataactgcagatgtaggagactctaccagagcgctatactttagagagaagtttaaggatagatactttaatgtaggcatagcagagcaagatatggtgaattttgctgctggcttggctgctgtaggaaaaaagcccgctatagttaactttggaatgttcttaatgagagcgtgggagcagataagaaatagtatagctagaatgaatctagacgtcaagatgtttgtaacacacaccggatacagtgaccacggtgatggttcgagtcatcaagttctcgaagatatagcgctaatgcgtgtattaccaaacatgaaagtagtagtaccagcagatcctaaggatattgaaagaagcttaccagttataattaatgaggaaaggggtccattgtattataggataggtagagaatattcaccaccaatcactataggacaagaatacgaattcaagattggtaaagcttatgtgattaaggatgggagtgacttagccataataggagcaggcgttgttttgtgggatgcactaaaggcggctgaagaattagagaaattaggaattagcgtagcagttataaatttattctcaataaagcctattgacgaaaatacaatagaatattatgctagaaaggctggtaagataattactattgaggaacatagcatatatggaggtattggttctgccgttgcagaggttacggctaggcgttatccagtacccataagatttgtaggtgctacgacttttggaagatctgctagaagccaaagggatctactagattactataatataaactataaaacaattataagggaggcaattgatttattgaagtagatgactgaagaaataacacggctgagggaagaaatagataaggtagacgagcagttagtaaagttactctcatatagattagaattatctagaaaaatagggaaagctaagtcgaattctaatataagtgttactgacgagaatagggaaatgaaagttagagaaaaatggattgctaacgcaaaaaagtataatattccaaatagtctggttgaatctatattgcctttgattttttcttattctaaactagttcagattaacccaggagagaaagaaagagtagtaatatatggatatggcggaatggctaaatcgatcgtttctattctttcattagctgggcatgaagtatcgattactggaagagatttaagtaaagcggagatgttagctaatcaatttaaatgtgtaagtatgtccttattaaaagcaatagattggggggatataattatatttgcaatacctcctagtgcaatattaaataattccgatgaattattttcaaaggcacttaaagataagattgttatggatattagttcttctaaatttaaaatatttggcttcctagaagaattatctaggaaactagagtttaggtatatttctacacatccacttttcggtcctattgaataccctattggagagagagttgtaattataccttccaaaactagttctaatgatgatgtcatgaaggtggagaatttctggaggaaaagtggtttagtacccgtcataactgatgttgaaactcatgaaaaagcaatggctattgttcaagttctaacgcattattatcttctgggtttatcaaacgcaattgatactttatcgttagagttaggtgtagattacagtaatttccatactacaaactttagagaattaaacaagattttaaagcgggttaaagatctaaaaaatgtaattactgaaatacaaaatcaaaacccttattcttataaagttagaaatataggtttagaggagcttaaaaaaattaaagaagaattagaaggaggtaaatagaatgatcttatatgtccttaaggatagagctgattactctatactaatagaaaagctaaatgaaaactcagcatctttcaagatattaaacctatatggtaaaaacttaatattagcatggccagatcagaacgtgaaaggtatcattgataatagtatagaaatggctgtggaagtaaagaaaagctatgtattagctggtaatgattggaaaaagcaaccaacagtggtaaatgtaaaagatgtagaaattggaagcaaaaaggtaatagtagctgcaggtccttgtgcagtagaaaatgaagaacaagtccttactactgctaaggctgtaaaaagggctggagcatcattacttagaggaggggcttacaaacctaggacaagtccatattccttccaaggtctcggagaagaaggggtgaaaatcttgaggagagtaggagatgaagtaggcttacctattgtcacagaaataatggatacaagagattccaatatatttagccaatatgttgatatgatacagattggagccagaaacgcacagaacttctctttattgaaggaagttggaaagttaggtaaaccagtactacttaagcgaggtatgggaaatacagtagaggaatggcttcaagctgcagagtacattttactagagggaaatggcaatacagtattatgcgaaagaggaataagaacatttgaaaagtcaactaggtttacgttagatataggtgggatggtagctgctaaactaatgacacatttgcccatctgtgctgatccaagtcatcctgcgggaaaaagagaattggtacactctttagcactagctgcagtcgctgctggtgcggatatgttattaattgaagttcatccacatccagaaaaggcattaagtgattcagagcaacaacttacaccggaatcattcgaagttctaatgaatcgaattagaacgctagctagagctttagggagagatgcatgagggaaatcttagaagatatttgttgctctgaagtaagagtagtagtaggagagggatcactttcaaaattatctaagattaaagacaataacgctgcagttatctattcaagaaaaattagtatagcagataaaattaataaatatttaccaaatgcatacttcatcccaattaatgatggtgaaagtactaaagaattatctagtgtaatatctttagtagaaaagctatttgaaaagaatttcgataggggggattatattataggtgttggtggtggaacggtaactgatgtagctggtttcttagcatctatatatttaagaggattaaatctgataaacgtaccaacgaccttcttaggcatggtcgatgcagcaatagggggtaagaatggagtaaatttcaataatataaagaacttaattggaacattctatcaaccaagtatgataatttccgatttagaatttttggaaactctaccaatagaagaactaaagaagggattagctgaagtaattaaatatggcttaactttagataaagaattatatgattacttgtctttaaataaggagaagatactaaataaagataaacaagcattagaagatataatctttagatctacacttgataaactaagtattgtaaaagaagatgagagagagactaaaggaatacgaatagttctaaatttcggccatacgataggtcatgctatagaagctggatcctcttttaatgttccacatggctacgctatctctgtaggaatggtttgtgaggctaagatggcggaagagttaggttatgcagaggaaggagtagtagaagatgtgttatggctattacagatttatggtttaccttacgatatatctcaaatagatgccccagtagatcttaaactagcattaaatgctattaatatggataaaaaacataggaaagatgtaattttgataccgtttcctactagaataggtagctggaaaaaagttgaagttcctctagataccgtaaaggggtttgccgaacaatgcttgaagaaataaattatgatactaagttattcggtctaataggtaaaaacataaagtacacgctatccccttatattcataatttctcatttagaacactaggaataaatgcagtttatctagtttttgatctcgacgaaatgaaattcaagcgtagtattagtgggatattggaaattgcagaaggacttaatgttacgataccgtataaggatgaagtaatgaaatatttggataatactgatacgcactccacgagaattcaagctgtaaatacaatatataaaaaaagtggttataacactgattatttagcaataaaaaatcttgtaagaaagaagattaagaatgtatctggctacgaatgttacatatatggggctggaggtgcagcaaaagcagcagcttttgcgttatctgaattaggatgctctagtattagtattgtgaatagaacaaaatcaagggcttatgagttagctgaattattaaataagaacggttataacgcgtcaattaaagagaattgcaacattacaaataatatacttattgtcaatagcactcctaattcttctgtagtcccagaggactgtgttaaaaaatctgatcttgttatagaatttgtttatagaccagttgagactgagttaattaaaaatgctaaaaaatatggtatacaatatataaacggtctagaaattttagtgaatcaagctgtagaagcggagaagatatggtttaataagagtgtggcagatgaaaagattatagagtatctttatgccagggaactcgtttggtaaactatttagaataaccacttttggagagagccatggtcctgcagtaggtgtagtcatagacggtgttcctgccggtttaccattaactgttgaagatataaagttcgaattagaatttagaagaccaggtagactatacgtttctggaaggagagaaaaagatgagccggaaatattaagtgggatctttaataatagaactaccggatctccaatagcagttatagtacgaaatactgatgtaatatcaagtttttatgacgagattaaatataaaccaagaccaggacatgcagaccttccatttataatgaaatatggatatgaaaattgggattataggggaggtggaagagcaagtgctagagaaactgtaagtagagttatagctggtgcagtagctaagaagttacttatgctaacagatacttggatagctggccatcttagaagtttaggcccagaagagttgagtgaagaggtaacatttgaggaggttctatgctcaaaatatagcccagtaagagctagtaaaaaagaccttgaggaaaaatatgaagcattaataaagaaagctactcaagaaggggatagctatggcggaatagctgaagtaatagccaagaatccaccaataggtttaggagaaccagtctttgataagatgaaagctgaattggctaaagcaataatgtcgatccctgctgtgatgggcttcgagtatggtttaggttttattgctagtaaaatgaaaggaagtgaggctaatgacgagattataagaaagaataatagaattggctggaagtacaattacgctggaggcattttaggtggtttaacaaatggtgaagatcttatagtgagatgtgcatttaaacctactagctcgattagaaagcctcaaaagaccatagatttaaggaacttagaggagagttatatttcagtaattggcagacacgacccagctgtagcaattaggggagttactgttgtagaatcaatggtagcgttaaccatagtagaccatgcaatgcgtgcaggagctattccactagttaaacttacagaggaccaagctaatacaatacagcaacgttgggagaggtatgtgaaatcatgcaagcctatggaggagtctcaatcgtaaacgcactaccatcttggtatggctcatctatggcaatcaatttgaaggtaaaagtagaaattagagaaggtaagagagtttattctcaagagagtgaactaattaagaccattcttaattactttaaagaaaaatattcaataccggatattgaagttgatattgaatctgaacttccacaaaagagtggactaaaaagcagtagtgcagtttctgtagccctaatagcggagattgcaaagcaatatgatctaaggaatattaaccctccaatattatctgcgatactttcactgaaagctggagtgtcatataccggggcacttgatgatgcagttgcatcatattgtggaggaatagcattcacttataataagatgtttagaatagtaaagttagagaatcttgaggataatttatcgatcctcatattagctaagggagggagacaaaaacctgttaatctaaacgagctaagaaaatatagtcacgtctttgaagaaatttttaagatagcacttaaggattacttgactgctatgaagatgaatggaatattgattgctaatattttaggctattcattagaaccaatagaaattgcactgaaaaaaggagcgttagctgccgggattagtggaaatgggccttcatattttgcagtttctaagaatggagaagaaggtccgatatacgaaagtcttaagaaatttggagatgttattatagttaggcctgtaagtcttgattgtaaagatttatccatcaaagattagtggaataataaaagctcctcaatcaaaaagtctagctattaggttaatttttctttcacttttcactagagtatatcttcataacttagttctatcggaagatgttatagacgctataaaatcagtaagagcattaggagtaaaggtaaaaaacaattctgaatttatacctccagagaaattagaaattaaggagaggtttataaaattaaaaggttccgctactactcttagaatgcttattccaatattagccgcaataggcggagaagtgacaattgatgcagatgagagtttaagaaggagacctttaaacagaatcgtacaagcattaagtaactacggtatatccttttcttcttacagtttgcctttgactatcacgggaaagttaagtagtaatgagataaagatttctggtgatgagagtagtcaatatatttctggcttaatatacgcacttcatattctaaatggcggtagtattgaaatattgccccccatttcatctaaaagttatattctgcttacaatagatttatttaagagatttggttctgatgttaagttttatggtagtaagattcatgttaatcccaataatttggttgaatttcaaggcgaagtggcgggagattatggtttagcctcgttttacgcgctttctgcattagttagtggtggaggaattacaataactaatttgtgggagccgaaggaatattttggtgatcatagtattgttaaaatatttagtgagatgggcgcttccagtgaatataaagacggtagatggtttgtcaaggctaaagataaatattctcccataaaaattgatatagatgacgcacctgacctggctatgacaattgcgggattatctgcaatagcggagggaacaagtgaaattatagggatcgaaagattgaggattaaggaaagtgatagaattgaaagtataaggaaaatcttaggattatatggtgtaggtagtgaagtaaagtataattctattctgatattcggaattaacaagggtatgttaaactctccagttacagactgtttgaatgatcacagagttgctatgatgtcgtcagccttagctttagtgaatggtggggtaattacatcagctgaatgtgtaggtaaaagtaatcctaattactggcaagatttattatcactaaatgcgaagatttctattgaatgagaccattaattgtagcttcattaccaattaaaaagatagaagacttaaaacttatagaaaattttttagatgcagatctaatagaactaagacttgattatctaagagaaagagaagtcagtttgatatctgactattatgaatttttagataaatataaaaagaagttaatagtaacgttaagagataaaggggagggaggaataaatcaattagcggatgaattaaagataaaaattttaaatgaactctacgagagacaatatctgtatgatatagaggtttcatttcttcaaaaatatgatataccatacgataataggatagtttctgtccactattttaattatcttccaactctagagaagataaaggaaattgttagcaagttttccgaaaaagcgttcagcgttaagattgcagttcctagtctaaaaggatataaggaggtactcttacctcttcttgaatatgaaaacgtaaccgtaattccaatgagtaataattctttagagaggatcgcagtgggtctactgggctcaaagttagtttattcgtacgcaattgaacctttagcacaagggcaactttactataaaaaagttatccagatttttaattatattaacgatataacaacttcatctttagttacttgaactctgtatacttttataggctttttggaagctccctttttaggttctccactatatatagaaaaatgagaaaagtgacattgacatactaattcttcttttatgataacgccatattttgaaagatcacaacctaaatgcgggcatttattatcaaaaacaaaaaatctatctactcctagataaaaaacgactaattcacgtccattaggcagtataatttttctcttttcaccagtcttaaaatcagttctacttatccttatatactccacgactttaattatgagctaacggagaaattaaagcaaactatgtgttaagcataaaaataagactcttatataataacataactttaatagagttattgctctaaaaggtaatgccaaaattcttccttataggtcatttaatcttgataatcaaatggcaagatttaaacataataggtgtagaataataaaaactgcttaaaagattatgaacaaacaatttataagattgggagcaaaataagtagattagaggaaatcgaagatgttagaaataagtgaggatcttaaagcaaagcttgattataga

SEQ ID NO: 12 - protein, 414 aa, Solfolobus solfataricus
MIVKIYPSKISGIIKAPQSKSLAIRLIFLSLFTRVYLHNLVLSEDVIDAIKSVRALGVKVKNNSEFIPPEKLEIKERFIKLKGSATTLRMLIPILAAIGGEVTIDADESLRRRPLNRIVQALSNYGISFSSYSLPLTITGKLSSNEIKISGDESSQYISGLIYALHILNGGSIEILPPISSKSYILLTIDLFKRFGSDVKFYGSKIHVNPNNLVEFQGEVAGDYGLASFYALSALVSGGGITITNLWEPKEYFGDHSIVKIFSEMGASSEYKDGRWFVKAKDKYSPIKIDIDDAPDLAMTIAGLSAIAEGTSEIIGIERLRIKESDRIESIRKILGLYGVGSEVKYNSILIFGINKGMLNSPVTDCLNDHRVAMMSSALALVNGGVITSAECVGKSNPNYWQDLLSLNAKISIE

SEQ ID NO: 13 - DNA, 1892 bp, Arthrobacter globiformis
  CDS (109)...(1417): Strain ATX21308
  misc_feature 1801: n= A, T, C or G
gggaccacatgctgctcctgatttcagggctgctgccggtatggaccagggtttagagagggacggcacgcatccgggcccttatcggaccaacgccaacagcggtcggtggccttggagcggggccagcacggccgatcacgtagactctttggagcttcgctcgaaaggatcaccatggaaactgatcgactagtgatcccaggatcgaaaagcatcaccaaccgggctttgcttttggctgccgcagcgaagggcacgtcggtcctggtgagaccattggtcagcgccgatacctcagcattcaaaactgcaattcaggccctcggtgccaacgtctcagccgacggtgacaattgggtcgttgaaggcctgggtcaggcaccccacctcgacgccgacatctggtgcgaggatgcaggtaccgtggcccggttcctccctccattcgtcgccgcaggacaggggaagttcaccgtcgacggaagcgagcagctgcggcggcgcccgcttcggcccctggtcgacggcatccgccacctgggcgcccgcgtctcctccgagcagctgcccctaacaattgaagcgagcgggctggcaggcggggagtacgaaattgaagcccatcagagcagccagttcgcctccggcctgatcatggccgccccgtacgcgcgacaaggcctgcgtgtgcggataccaaatcccgtgagccagccctacctcacgatgacactgcggatgatgagggacttcggccttgagaccagcaccgacggagccaccgtcagcgtccctcccgggcgctacacagcccggcggtatgaaattgaaccggacgcgtcaactgcgtcgtacttcgccgccgcttccgccgtctctggccgaagcttcgaattccagggccttggcacagacagcatccaaggcgacacgtcattcttcaatgtacttgggcggctcggtgcagaggtccactgggcacccaactcggtcaccatatccggaccggaaaggctgaacggcgacattgaagtggatatgggcgagatatcggacaccttcatgacactcgcggcgattgcccctctagccgatggacccatcacgataaccaacattggccatgcacggttgaaggaatccgaccgcatctcggcgatggaaaccaacctgcgaacgctcggtgtacaaaccgacgtcggacacgactggatgcgaatctacccctctaccccgcacggcggcagagtcaattgccaccgggaccacaggatcgccatggcgttttcaatcctgggactgcgagtggacgggattaccctcgacgaccctcaatgtgtcgggaagacctttcctggcttcttcgactaccttggacgccttttccccgaaaaggcgcttacgctccccggctagtgacttcctctccggcggacgctaggcatcggaaaacgaatcctgacatgaccgacctcctcgcgtcacggcgtgtctgccggtacccaagcattctgccttagccgcttccgcggccccttatgctttctggttgtccagattttcatccgggatgttgcctgaccttgagcagggcaatcagctgttcagcactgtcaatggtgtgggccctgaaggcggcttcgatggctgccacgtcggcggctctcatcgctgtcacgacacgcagatgcgcttcataggcacgttcaggatccgccctcgtcgcctgatcctgagccaaggcaatagttagatgtgcctccgttggcggccagagccgaagcaataaggagttttncgaggccacccagattccccgggtggaaggcgatatgggcttcatgctgaactatggggtccggatggaagtgacttttcaactctgccca

SEQ ID NO: 14 - protein, 436 aa, Arthrobacter globiformis
  VARIANT (0)...(0): Strain ATX21308
MALERGQHGRSRRLFGASLERITMETDRLVIPGSKSITNRALLLAAAAKGTSVLVRPLVSADTSAFKTAIQALGANVSADGDNWVVEGLGQAPHLDADIWCEDAGTVARFLPPFVAAGQGKFTVDGSEQLRRRPLRPLVDGIRHLGARVSSEQLPLTIEASGLAGGEYEIEAHQSSQFASGLIMAAPYARQGLRVRIPNPVSQPYLTMTLRMMRDFGLETSTDGATVSVPPGRYTARRYEIEPDASTASYFAAASAVSGRSFEFQGLGTDSIQGDTSFFNVLGRLGAEVHWAPNSVTISGPERLNGDIEVDMGEISDTFMTLAAIAPLADGPITITNIGHARLKESDRISAMETNLRTLGVQTDVGHDWMRIYPSTPHGGRVNCHRDHRIAMAFSILGLRVDGITLDDPQCVGKTFPGFFDYLGRLFPEKALTLPG

SEQ ID NO: 15 - protein, 425 aa, Agrobacterium tumifaciens
MIELTITPPGHPLSGKVEPPGSKSITNRALLLAGLAKGKSRLTGALKSDDTLYMAEALREMGVKVTEPDATTFVVESSGGLHQPEKPLFLGNAGTATRFLTAAAALVDGAVIIDGDEHMRKRPIMPLVEALRSLGVEAEAPTGCPPVTVCGKGTGFPKGSVTIDANLSSQYVSALLMAAACGDKPVDIILKGEEIGAKGYIDLTTSAMEAFGAKVERVSNAIWRVHPTGYTATDFHIEPDASAATYLWGAELLTGGAIDIGTPADKFTQPDAKAHEVMAQFPHLPAEIDGSQMQDAIPTIAVLAAFNETPVRFVGIANLRVKECDRIRAVSLGLNEIRDGLAHEEGDDLIVHSDPSLAGQTVNASIDTFADHRIAMSFALAALKIGGIAIQNPACVGKTYPGYWKALASLGVEYSEKETAAEPQH

SEQ ID NO: 16 - protein, 418 aa, Pseudomonas syringae
  VARIANT (0)...(0): pv phaseolicolla strain 1448a
MRPQATLTVLPVERPLVGRVSPPGSKSITNRALLLAGLAKGTSRLTGALKSDDTRVMSEALRLMGVQVDEPDDSTFVVTSSGHWQAPQQALFLGNAGTATRFLTAALANFEGDFVVDGDEYMRKRPIGPLVDALQRMGVEVSAPSGCPPVAIKGKGGLEAGRIEIDGNLSSQYVSALLMAGACGKGPVEVALTGSEIGARGYVDLTLAAMQAFGAEVQAIGETAWKVSATGYRATDFHIEPDASAATYLWAAQALTEGDIDLGVASDAFTQPDALASQIIASFPNMPAVIDGSQMQDAIPTLAVLAAFNRQPVRFVGIANLRVKECDRISALSHGLCAIAPGLAVEEGDDLLVHANPALAGTTVDALIDTHSDHRIAMCFALAGLKIAGIRILDPDCVGKTYPGYWDALASLGVRVQR

SEQ ID NO: 17 - protein, 444 aa, Ochrobactrum/Brucella
MACLPDDSGPHVGHSTPPCLDQEPCTLSSQKTVTVTPPNFPLTGKVAPPGSKSITNRALLLAALAKGTSRLSGALKSDDTRHMSVALRQMGVTIDEPDDTTFVVTSQGSLQLPAQPLFLGNAGTAMRFLTAAVATVQGTVVLDGDEYMQKRPIGPLLATLGQNGIQVDSPTGCPPVTVHGAGKVQARRFEIDGGLSSQYVSALLMLAACGEAPIEVALTGKDIGARGYVDLTLDCMRAFGAQVDIVDDTTWRVAPTGYTAHDYLIEPDASAATYLWAAEVLTGGRIDIGVAAQDFTQPDAKAQAVIAQFPNMQATVVGSQMQDAIPTLAVLAAFNNTPVRFTELANLRVKECDRVQALHDGLNEIRPGLATIEGDDLLVASDPALAGTACTALIDTHADHRIAMCFALAGLKVSGIRIQDPDCVAKTYPDYWKALASLGVHLSY

SEQ ID NO: 18 - protein, 418 aa, Pseudomonas syringae
  VARIANT (0)...(0): Strain DC3000 EPSPS Gene
MRPQATLTVMPVERPLVGRVSPPGSKSITNRALLLAGLAKGTSRLTGALKSDDTRVMSEALRLMGVQVDEPDDSTFVVTSSGHWQAPQQALFLGNAGTATRFLTAALANFEGDFVVDGDEYMRKRPIGPLVDALQRMGVEISAPSGCPPVAIKGKGGLQAGRIEIDGNLSSQYVSALLMAGACGKGSLEVALTGSEIGARGYVDLTLAAMQAFGAEVQAIGDAAWKVSATGYHATDFHIEPDASAATYLWAAQALTEGNIDLGVASDAFTQPDALASQIIDSFPNMPAVIDGSQMQDAIPTLAVLAAFNRQPVRFVGIANLRVKECDRISALCDGLCAIAPGLAVEEGDDLIVHANPALAGTTVNALIDTHSDHRIAMCFALAGLKIKGIHIQDPDCVAKTYPGYWDALASLGVSVQR

SEQ ID NO: 19 - protein, 418 aa, Pseudomonas syringae
  VARIANT (0)...(0): pv syringae strain B728a
MRPQATLTVLPVERPLVGRVSPPGSKSITNRALLLAGLAKGTSRLTGALKSDDTRVMSEALRLMGVQVDEPDDSTFVVTSSGHWQAPQQALFLGNAGTATRFLTAALANFEGDFVVDGDEYMRKRPIGPLVDALQRMGVEVSAPSGCPPVAIKGKGGLEAGRIEIDGNLSSQYVSALLMAGACGKGPVEVALTGSEIGARGYLDLTLAAMRAFGAEVQAIGDAAWKVSATGYRATDFHIEPDASAATYLWAAQALTEGAIDLGVASNAFTQPDALASQIIASFPNMPAVIDGSQMQDAIPTLAVLAAFNRQPVRFVGIANLRVKECDRISALSNGLCAIAPGLAVEEGDDLIVTANPTLAGTTVDALIDTHSDHRIAMCFALAGLKIAGIRILDPDCVAKTYPGYWDALASLGVSVQR

SEQ ID NO: 20 - protein, 419 aa, Brevundomonas vesicularis
MMMGRAKLTIIPPGKPLTGRAMPPGSKSITNRALLLAGLAKGTSRLTGALKSDDTRYMAEALRAMGVTIDEPDDTTFIVKGSGKLQPPAAPLFLGNAGTATRFLTAAAALVDGKVIVDGDAHMRKRPIGPLVDALRSLGIDASAETGCPPVTINGTGRFEASRVQIDGGLSSQYVSALLMMAAGGDRAVDVELLGEHIGALGYIDLTVAAMRAFGAKVERVSPVAWRVEPTGYHAADFVIEPDASAATYLWAAEVLSGGKIDLGTPAEQFSQPDAKAYDLISKFPHLPAVIDGSQMQDAIPTLAVLAAFNEMPVRFVGIENLRVKECDRIRALSSGLSRIVPNLGTEEGDDLIIASDPSLAGKILTAEIDSFADHRIAMSFALAGLKIGGITILDPDCVAKTFPSYWNVLSSLGVAYED

SEQ ID NO: 21 - protein, 425 aa, Agroibacterium tumifaciens
  VARIANT (0)...(0): Strain C58 EPSPS
MIELTITPPGHPLSGKVEPPGSKSITNRALLLAGLAKGKSHLSGALKSDDTLYMAEALREMGVKVTEPDATTFVVEGTGVLQQPEKPLFLGNAGTATRFLTAAGALVDGAVIIDGDEHMRKRPILPLVQALRALGVEADAPTGCPPVTVRGKGMGFPKGSVTIDANLSSQYVSALLMAAACGDKPVDIILKGEEIGAKGYIDLTTSAMEAFGAKVERVSNAIWRVHPTGYTATDFHIEPDASAATYLWGAELLTGGAIDIGTPADKFTQPDAKAYEVMAQFPHLPAEIDGSQMQDAIPTIAVIAAFNETPVRFVGIANLRVKECDRIRAVSLGLNEIREGLAHEEGDDLIVHADPSLAGQTVDASIDTFADHRIAMSFALAALKIGGIAIQNPACVAKTYPGYWKALASLGVDYTEKESAAEPQH

SEQ ID NO: 22 - protein, 427 aa, Escherichia coli
MESLTLQPIARVDGTINLPGSKSVSNRALLLAALAHGKTVLTNLLDSDDVRHMLNALTALGVSYTLSADRTRCEIIGNGGPLHAEGALELFLGNAGTAMRPLAAALCLGSNDIVLTGEPRMKERPIGHLVDALRLGGAKITYLEQENYPPLRLQGGFTGGNVDVDGSVSSQFLTALLMTAPLAPEDTVIRIKGDLVSKPYIDITLNLMKTFGVEIENQHYQQFVVKGGQSYQSPGTYLVEGDASSASYFLAAAAIKGGTVKVTGIGRNSMQGDIRFADVLEKMGATICWGDDYISCTRGELNAIDMDMNHIPDAAMTIATAALFAKGTTTLRNIYNWRVKETDRLFAMATELRKVGAEVEEGHDYIRITPPEKLNFAEIATYNDHRMAMCFSLVALSDTPVTILDPKCTAKTFPDYFEQLARISQAA

SEQ ID NO: 23 - protein, 423 aa, Salmonella typhimurium
MESLTLQPIARVDGAINLPGSKSVSNRALLLAALACGKTALTNLLDSDDVRHMLNALSALGINYTLSADRTRCDITGNGGALRAPGALELFLGNAGTAMRPLAAALCLGQNEIVLTGEPRMKERPIGHLVDSLRQGGANIDYLEQENYPPLRLRGGFGGDIEVDGSVSSQFLTALLMTAPLAPKDTIIRVKGELVSKPYIDITLNLMKTFGVEIANHHYQQFVVKGGQQYHSGRYLVEGDASSASYFLAAGAIKGGTVKVTGIGRKSMQGDIRFADVLEKMGATITWGDDFIACTRGELHAIDMDMNHIPDAAMTIATTALFAKGTTTLRNIYNWRVKETDRLFAMATELRKVGAEVEEGHDYIRITPPAKLQHADIGNDHRMAMCFSLVALSDTPVTILDPKCTAKTFPDYFEQLARMSTPA

SEQ ID NO: 24 - protein, 444 aa, Zea mays
AGAEEIVLQPIKEISGTVKLPGSKSLSNRILLLAALSEGTTVVDNLLNSEDVHYMLGALRTLGLSVEADKAAKRAVVVGCGGKFPVEDAKEEVQLFLGNAGTAMRPLTAAVTAAGGNATYVLDGVPRMRERPIGDLVVGLKQLGADVDCFLGTDCPPVRVNGIGGLPGGKVKLSGSISSQYLSALLMAAPLALGDVEIEIIDKLISIPYVEMTLRLMERFGVKAEHSDSWDRFYIKGGQKYKSPKNAYVEGDASSASYFLAGAAITGGTVTVEGCGTTSLQGDVKFAEVLEMMGAKVTWTETSVTVTGPPREPFGRKHLKAIDVNMNKMPDVAMTLAVVALFADGPTAIRDVASWRVKETERMVAIRTELTKLGASVEEGPDYCIITPPEKLNVTAIDTYDDHRMAMAFSLAACAEVPVTIRDPGCTRKTFPDYFDVLSTFVKN

SEQ ID NO: 25 - protein, 455 aa, Agrobacterium sp. CP4
MSHGASSRPATARKSSGLSGTVRIPGDKSISHRSFMFGGLASGETRITGLLEGEDVINTGKAMQAMGARIRKEGDTWIIDGVGNGGLLAPEAPLDFGNAATGCRLTMGLVGVYDFDSTFIGDASLTKRPMGRVLNPLREMGVQVKSEDGDRLPVTLRGPKTPTPITYRVPMASAQVKSAVLLAGLNTPGITTVIEPIMTRDHTEKMLQGFGANLTVETDADGVRTIRLEGRGKLTGQVIDVPGDPSSTAFPLVAALLVPGSDVTILNVLMNPTRTGLILTLQEMGADIEVINPRLAGGEDVADLRVRSSTLKGVTVPEDRAPSMIDEYPILAVAAAFAEGATVMNGLEELRVKESDRLSAVANGLKLNGVDCDEGETSLVVRGRPDGKGLGNASGAAVATHLDHRIAMSFLVMGLVSENPVTVDDATMIATSFPEFMDLMAGLGAKIELSDTKAA

SEQ ID NO: 26 - protein, 428 aa, Bacillus subtilis
MKRDKVQTLHGEIHIPGDKSISHRSVMFGALAAGTTTVKNFLPGADCLSTIDCFRKMGVHIEQSSSDVVIHGKGIDALKEPESLLDVGNSGTTIRLMLGILAGRPFYSAVAGDESIAKRPMKRVTEPLKKMGAKIDGRAGGEFTPLSVSGASLKGIDYVSPVASAQIKSAVLLAGLQAEGTTTVTEPHKSRDHTERMLSAFGVKLSEDQTSVSIAGGQKLTAADIFVPGDISSAAFFLAAGAMVPNSRIVLKNVGLNPTRTGIIDVLQNMGAKLEIKPSADSGAEPYGDLIIETSSLKAVEIGGDIIPRLIDEIPIIALLATQAEGTTVIKDAAELKVKETNRIDTVVSELRKLGAEIEPTADGMKVYGKQTLKGGAAVSSHGDHRIGMMLGIASCITEEPIEIEHTDAIHVSYPTFFEHLNKLSKKS

SEQ ID NO: 27 - protein, 427 aa, K. pneumoniae
MESLTLQPIARVDGTVNLPGSKSVSNRALLLAALARGTTVLTNLLDSDDVRHMLNALSALGVHYVLSSDRTRCEVTGTGGPLQAGSALELFLGNAGTAMRPLAAALCLGSNDIVLTGEPRMKERPIGHLVDALRQGGAQIDYLEQENYPPLRLRGGFTGGDVEVDGSVSSQFLTALLMASPLAPQDTVIAIKGELVSRPYIDITLHLMKTFGVEVENQAYQRFIVRGNQQYQSPGDYLVEGDASSASYFLAAGAIKGGTVKVTGIGRNSVQGDIRFADVLEKMGATVTWGEDYIACTRGELNAIDMDMNHIPDAAMTIATAALFARGTTTLRNIYNWRVKETDRLFAMATELRKVGAEVEEGEDYIRITPPLTLQFAEIGTYNDHRMAMCFSLVALSDTPVTILDPKCTAKTFPDYFGQLARISTLA

SEQ ID NO: 28 - protein, 439 aa, Clostridium tetani
MHKEETFNQCALTINGYKSEVKKTYELPGDKSVGHRSLLIGALPKGEYKIRNFPQSRDCLTTLKIMEELGVKVKVLKDYILVNSPGYENFKKKIDYIDCGNSGTTSRLIAGILAGVGVETNLVGDKSLSIRPMKRIVDPLNSMGANIEMEKDHMPLIFKGNGELKGIDYTMEIASAQVKSCILLAGFLSEGVTKVRELSPTRDHTERMLKYIEGNIKIENKEIEIENSTIKSKDIYVPGDISSAAYIIACAILGEDCEIILENVLLNENRRKYLDLLKKMGANLKYLEKNQCNGEHVGNILVKSSFLKGISIGKEITPYIIDEIPIISLIASFAEGKTIFENVEELKYKESDRIKAIMVNLKSLGVKTELVENNLIIYGGLSKINKEINIRTFNDHRIALTFLCSAMRNSEKTYIDNWDCVAISFPNSLNYFKDFFRIN

SEQ ID NO: 29 - protein, 6 aa, Artificial Sequence
  Conserved Domains
  VARIANT 3: Xaa= Gly, Ser, Ala or Asn
  VARIANT 4: Xaa= Asn or Glu
DCXXSG

SEQ ID NO: 30 - protein, 6 aa, Artificial Sequence
  Conserved Domains
  VARIANT 3: Xaa= Ala or Arg
  VARIANT 4: Xaa= Asn or Glu
DAXXSG

SEQ ID NO: 31 - protein, 6 aa, Artificial Sequence
  Conserved Domains
  VARIANT 2: Xaa= Gly, Asn, or Glu
KLKXSA

SEQ ID NO: 32 - protein, 6 aa, Artificial Sequence
  Conserved Domains
WCEDAG

SEQ ID NO: 33 - protein, 10 aa, Artificial Sequence
  Conserved Domains
  VARIANT 3, 4, 7,  9: Xaa= Any amino acid
  VARIANT 8: Xaa= Ser or Thr
DCXXSGXXXR

SEQ ID NO: 34 - protein, 10 aa, Artificial Sequence
  Conserved Domains
  VARIANT 1, 3, 4, 7, 9: Xaa= Any amino acid
  VARIANT 8: Xaa= Ser or Thr
XCXXSGXXXR

SEQ ID NO: 35 - protein, 2 aa, Artificial Sequence
  Conserved Domains
  VARIANT 2: Xaa= Ile or Leu
PX

SEQ ID NO: 36 - protein, 10 aa, Artificial Sequence
  Conserved Domains
  VARIANT 3: Xaa= Ser or Thr
  VARIANT 4: Xaa= Gln or Asp
  VARIANT 8: Xaa= Ala, Leu, Met, Ile or Val
  VARIANT 9: Xaa= Phe, Ala, Leu, Met, Ile or Val
DAXXCPDXXP

SEQ ID NO: 37 - protein, 2 aa, Artificial Sequence
  Conserved Domains
LK

SEQ ID NO: 38 - DNA, 1320 bp, Clostridium tetani
  CDS (1)...(1320)
atgcataaggaagaaacttttaaccagtgtgcacttactattaatggatacaagtccgaggttaaaaagacctatgaacttccaggtgataaatctgtaggtcataggtctcttttaattggagccttgccaaaaggagaatataaaataagaaattttcctcaaagtagagattgtttaactactttgaaaataatggaagagctaggtgtgaaagttaaagttcttaaagattatatattagtaaactcaccggggtatgaaaattttaaaaagaaaattgattatatagactgtggaaattctggaactacttcaaggcttatagcaggtatattagcaggtgtaggagtggaaactaatttagtaggtgataaatccctctctataagacctatgaaaagaatagtagaccctctaaattctatgggagctaatatagagatggaaaaagatcatatgcccttaatttttaaaggtaatggagaactaaagggtattgattatactatggaaattgcctctgcccaggtgaaatcctgcattttattagctggatttttatcagaaggtgttacaaaggtaagagaattaagtcctacaagagatcacacagaaagaatgttaaaatacatagaagggaatataaaaatagaaaataaagaaatagaaatcgaaaattctaccataaagagtaaagatatttatgttccaggagatatatcttcagcagcatatattatagcctgtgccatattaggagaagactgtgaaattattttagaaaatgtattgttgaatgagaatagaagaaaatacttggacttattaaagaaaatgggagctaacttaaagtacttagagaaaaatcagtgtaatggagaacatgtaggtaatattttagttaagagtagttttttaaagggtataagtataggaaaagaaattacgccttatataatagatgaaatacctataatatcccttatagcctcctttgcagaaggaaagaccatatttgaaaatgtagaggagttaaagtacaaagaaagtgatagaataaaggcaattatggtgaatttaaagtcacttggggtaaaaacagaattagtagaaaataatttaattatctatggaggactttctaagataaataaagaaattaatattagaacctttaatgatcacagaatagcattaacttttttgtgttcagctatgagaaatagtgaaaaaacttatatagataattgggattgtgtagccatatcctttccaaattctttgaattattttaaggattttttcagaataaattaa

SEQ ID NO: 39 - protein, 439 aa, Clostridium tetani; identical to SEQ ID NO: 28

SEQ ID NO: 40 - DNA, 1293 bp, Methanosarcina mazei
  CDS (1)...(1293)
atgcgcgcctcaattagcaaatcctcaatcaaaggggaggtctttgcccctccttcaaagagttacacccacagggctataactctcgcagccctttcaaaagaatcgatcattcaccgtcccctcctttccgctgatactcttgctacaatcagagcttctgagatgttcggagccgcggttagacgggagaaagaaaatctcatcatccagggatctaatggaaagcccggtattcctgatgatgtaattgatgccgcaaattcagggacaaccctccgctttatgacagcaatagcaggcttaactgacggaatcactgtacttacaggagactcatctcttcgcacgcgtccaaacggacctcttcttgaagttctcaacaggctgggagcaaaagcctgttctacgcgaggaaacgaaagagcgcctattgtggtcaaaggaggaattaagggatctgaagtggaaataagcggctcgatcagctcccagtttatctctgctcttcttatagcctgcccgcttgctgaaaacagcaccactctttccattataggaaaactgaagtcaagaccttatgttgacgtgaccatagaaatgctcgggctggcaggagtcaaaatccatacagatgataataacggcacgaaatttatcatccccggaaaacagaaatacgacctgaaacaatacacggttcccggagacttttcttctgcttcctacctgctagcagctgcagccatgcttgaaggctccgaaatcacagtcaaaaatctattcccttcaaaacagggagataaagtgattattgatactctcaaacagatgggagcagacataacatgggacatggaagctggcattgtgaccgtaagaggaggaagaaaattaaaagccattacctttgatgccggatcaacccctgaccttgtaccgactgttgccgtccttgcttcagttgccgaagggaccagcagaatagaaaacgccgagcatgtccgctataaagaaacagaccggcttcacgcccttgcgaccgagcttccgaaaatgggagtctccctcaaagaagaaatggacagcctgacaatcaccggagggactcttgagggagccgaagtccacggctgggacgaccaccggattgtgatgtctctagctatagcaggcatggttgcaggaaacacgatagttgacaccactgagtctgtatcgatatcctatcctgatttctttaaagatatgcgaaaccttggagcaaaagtcaaggagattcctgaagaataa

SEQ ID NO: 41 - protein, 430 aa, Methanosarcina mazei
MRASISKSSIKGEVFAPPSKSYTHRAITLAALSKESIIHRPLLSADTLATIRASEMFGAAVRREKENLIIQGSNGKPGIPDDVIDAANSGTTLRFMTAIAGLTDGITVLTGDSSLRTRPNGPLLEVLNRLGAKACSTRGNERAPIVVKGGIKGSEVEISGSISSQFISALLIACPLAENSTTLSIIGKLKSRPYVDVTIEMLGLAGVKIHTDDNNGTKFIIPGKQKYDLKQYTVPGDFSSASYLLAAAAMLEGSEITVKNLFPSKQGDKVIIDTLKQMGADITWDMEAGIVTVRGGRKLKAITFDAGSTPDLVPTVAVLASVAEGTSRIENAEHVRYKETDRLHALATELPKMGVSLKEEMDSLTITGGTLEGAEVHGWDDHRIVMSLAIAGMVAGNTIVDTTESVSISYPDFFKDMRNLGAKVKEIPEE

SEQ ID NO: 42 - DNA, 38 bp, Artificial Sequence
  Oligonucleotide primer
cagggatccgccatgaattgtgttaaaataaatccatg

SEQ ID NO: 43 - DNA, 32 bp, Artificial Sequence
  Oligonucleotide primer
cagggcgcgccttattcccccaaactccactc

SEQ ID NO: 44 - DNA, 35 bp, Artificial Sequence
  Oligonucleotide primer
cagggatccgccatgattgtaaagatttatccatc

SEQ ID NO: 45 - DNA, 36 bp, Artificial Sequence
  Oligonucleotide primer
cagggcgcgccggtctcattcaatagaaatcttcgc

SEQ ID NO: 46 - protein, 418 aa, Rhodobacter sphaeroides
LKGRAEIPGDKSISHRALILGAMAVGETRITGLLEGQDVLDTAKAMRAFGAEVIQHGPGAWSVHGVGVGGFTEPAEVIDCGNSGTGVRLVMGAMATSPLTATFTGDASLRKRPMGRVTDPLALFGTRAYGRKGGRLPMTLVGAADPVPVRYTVPVPSAQVKSAVLLAGLNAPGQTVVIEREATRDHSERMLRGFGAELSVETGPEGQVITLTGQPELRPQTVAVPRDPSSAAFPVCAALIVEGSEILVPGVSRNPTRDGLYVTLLEMGADIAFENEREEGGEPVADLRVRASALKGVEVPPERAPSMIDEYPILSVVAAFAEGLTIMRGVKELRVKESDRIDAMARGLEACGVRIEEDEDTLIVHGMGRVPGGATCATHLDHRIAMSFLVLGMAAEAPVTVDDGSPIATSFPAFIDLM

SEQ ID NO: 47 - protein, 424 aa, Chloroflexus aurantiacus
KRLRGVIEVPGDKSISHRSVLFNAIATGSAHITHFLPGADCLSTVACIRALGVTVEQPAERELIVHGVGLGGLREPADVLDCGNSGTTLRLLAGLLAGHPFFSVLTGDASLRSRPQRRIVVPLRAMGAQIDGRDDGDRAPLAIRGNRLRGGHYELSIASAQVKSALLLAALNAEQPLTLTGRIDSRDHTERMLAAMGLEITVTADQITIQPPSEATAPTALSLRVPGDPSSAAFWWVAAAIHPDAELVTPGVCLNPTRIGAIEVLQAMGADLTVMNERLEGSEPVGDVVVRSSSLRGTTIAGTLIPRLIDEIPVLAVAAACASGETVIRDAQELRAKETDRIATVAAGLSAMGAVVEPTADGMVIVGQPGQLQGTTLNSFHDHRLAMAWAIAAMVARGETTILEPAAAAVSYPEFWQTLAMVQE

SEQ ID NO: 48 - protein, 424 aa, Methanosarcina mazei
MRVSIDKSSIKGEVFAPPSKSYTHRAVTLAALSKESTVRHPLISADTLATVRASEMFGALVEREEDRLIIHGINGKPNVPDDVIDAANSGTTLRFMTAVAALTDGITVLTGDASLRTRPNGPLLEVLNRLGVKACSTRGNERAPLVVKGGLKGQDVSIDGSISSQFISALLITCPLAENSTILSITGKIKSRPYVDITLEMLELAGVKVHIDDSNGTRFIIPGKQKYDFKDYTVPGDFSSASYLLAAAAMTDGSEVTVKNLFPSKQGDKVIIETLKQMGADITWDKEAGNVTVKGGRQLKAITFDAGANPDLVPTVAVLAAVAKGTSRIENAEHVRYKETDRLRALATELPKLGVDLKEERDSLTITGGKLHGASVHGWDDHRIVMALSVAGIVAGNTKIDTTESASISYPEFFKDMRSLGAKI

SEQ ID NO: 49 - protein, 439 aa, Halobacterium sp. NRC-1
MPWAALLAGMHATVSPSRVRGRARAPPSKSYTHRALLAAGYADGETVVRDPLVSADTRATARAVELLGGAAARENGDWVVTGFGSRPAIPDAVIDCANSGTTMRLVTAAAALADGTTVLTGDESLRARPHGPLLDALSGLGGTARSTRGNGQAPLVVDGPVSGGSVALPGDVSSQFVTALLMAGAVTETGIETDLTTELKSAPYVDITLDVLDAFGVGASETAAGYRVRGGQAYAPSGAEYAVPGDFSSASYLLAAGALAAADGAAVVVEGMHPSAQGDAAIVDVLERMGADIDWDTESGVITVQRSELSGVEVGVADTPDLLPTIAVLGAAADGTTRITDAEHVRYKETDRVAAMAESLSKLGASVEERPDELVVRGGDTELSGASVDGRGDHRLVMALAVAGLVADGETTIAGSEHVDVSFPDFFEVLAGLGADTDG

SEQ ID NO: 50 - protein, 424 aa, Synechococcus SP. WH 8102
GGSLSGHVKVPGDKSISHRSLLFGAIAEGTTTIDGLLPAEDPISTAACLRAMGVLISPIEAAGLVTVEGVGLDGLQEPAEILDCGNSGTTMRLMLGLLAGRAGRHFVLDGDASLRRRPMRRVGQPLASMGADVRGRDGGNLAPLAVQGQSLRGTVIGTPVASAQVKSALLLAALTADGTTTVIEPAQSRDHSERMLRAFGADLQVGGEMGRHITVRPGNTLKGQQVVVPGDISSAAFWLVAGALVPGADLTIENVGLNPTRTGILEVLEQMNAQIEVLNRRDVAGEPVGDLRITHGPLQPFSIGEEIMPRLVDEVPILSVAACFCDGESRISGASELRVKETDRLAVMARQLKAMGAEIEEHEDGMTIHGGRPLKGAALDSETDHRVAMSLAVASLLASGDSTLQRSDAAAVSYPSFWDDLDRL

SEQ ID NO: 51 - protein, 322 aa, Archaeoglobus fulgidus
MAEFPKVRMRRLRKANLRWMFREARLSPENLITPIFVDENIKEKKPIESMPDYFRIPLEMVDKEVEECLEKDLRSFILFGIPSYKDETGSSAYDQNGVIQKAVRRIKAEFPDAVIVTDVCLCEYTTHGHCGVVKDGEIVNDETLPIIGKTAVSHAESGADIVAPSGMMDGMVKAIREALDAAGFESTPIMSYSAKYASNFYSPFRDAAESGFKFGDRRGYQMDIHNAREAMREIELDVKEGADIIMVKPALPYLDIIRMVRERFDLPLAAYNVSGEYSMIKAAIKNGWLSEEAIYEVLISIKRAGADLIITYHSKEIAEKLQ

SEQ ID NO: 52 - protein, 410 aa, Pyrococcus abyssi
MFGPVSVEMIIERVDEVRGKVKAPPSKSYTHRAYFLSLLADSPSKVMNPLISEDTIASLDAISKFGAQVNGNKIIPPQELTPGKIDARESGTTARISLAVASLARGTSVITGKGRLVERPFKPLVDALRSLKVKISGEKLPIAVEGGNPVGEYVKVDCSLSSQFGTAMLILASKIGLTVEMLNPVSRPYIEVTLKVMESFGIEFERNGFKVKVHPGIRGSKFHVPGDYSSASFFLAAGALYGKVKVSNLVKDDPQADARIIDILEEFGADVKVGRKYVVVERNEMKPINVDCSNFPDLFPILAVLASYAEGKSVITGRQLRLKESDRVKAVAVNLRKAGIKVKELPNGLEIVGGKPRGFTVESFNDHRIVMAMAILGLGAEGKTIIKDPHVVSKSYPSFFLDLRRVLNEG